(19) United States
(12) Patent Application Publication
(10) Pub. No.: US 2021/0018463 A1
(43) Pub. Date: Jan. 21, 2021

(54) VIRUS BIORESISTORS

(71) Applicants: The Regents of the University of California, Oakland, CA (US); Phagetech, Inc., Newport Beach, CA (US)

(72) Inventors: Reginald M. PENNER, Newport Beach, CA (US); Alana F. OGATA, Oakland, CA (US); Apurva BHASIN, Oakland, CA (US); Gregory A. WEISS, Irvine, CA (US); Phillip TAM, Newport Beach, CA (US); Jeffrey Scott BRIGGS, Newport Beach, CA (US); Marie YAP-TRUE, Newport Beach, CA (US); Aisha ATTAR, Irvine, CA (US); Shae Victoria PATTERSON, Newport Beach, CA (US)

(21) Appl. No.: 17/043,191

(22) PCT Filed: Mar. 29, 2019
(86) PCT No.: PCT/US19/24939

§ 371 Date: Sep. 29, 2020

Related U.S. Application Data
(60) Provisional application No. 62/650,059, filed on Mar. 29, 2018.

(51) Int. Cl. G01N 27/414; C12N 7/00; G01N 27/327; G01N 27/02

(52) U.S. Cl. CPC G01N 27/4145; C12N 7/00; C12N 2795/14131; G01N 27/026; G01N 27/3276

(57) ABSTRACT

Provided herein are, inter alia, biosensors and electrochemical cells comprising electronically conductive polymers and viral particles; diagnostic kits; and methods of detecting compounds in samples.

### CROSS-REFERENCES TO RELATED APPLICATIONS

This application claims priority to U.S. Application No. 62/650,059 filed Mar. 29, 2018, which is incorporated herein by reference in entirety and for all purposes

### STATEMENT AS TO RIGHTS TO INVENTIONS MADE UNDER FEDERALLY SPONSORED RESEARCH AND DEVELOPMENT

This invention was made with government support under grant no. 1R33CA206955-01 awarded by the National Cancer Institute of the National Institutes of Health, and grant no. 1803314 awarded by the National Science Foundation. The government has certain rights in the invention.

### BACKGROUND

Biosensor technologies that enable the rapid measurement of disease biomarkers in unprocessed biological samples, including blood, urine, saliva, lacrimal fluid, nipple aspirate fluid, and cerebrospinal fluids, remain elusive and highly sought. The ultimate goal is devices that can be used with minimal training by physicians and patients to provide actionable information at the point-of-care (PoC) (Gubala et al (2012) Anal. Chem. 84:487-515; Soper et al (2006) Biosens. Bioelectron. 21:1932-1942; Luo et al (2013) Chem. Soc. Rev. 42:5944-5962). In addition to simplicity, analysis speed and sensitivity are critically important metrics for PoC biosensors but the technology must also provide for sensor-to-sensor reproducibility, manufacturability, and low cost.

A new approach to point of care detection of protein disease markers involves the use of virus particles, rather than antibodies, within a bioaffinity capture layer. Relative to antibodies, virus particles have several advantages that make them attractive for emerging PoC sensor technologies: First, virus particles can be engineered to bind virtually any protein, including toxic proteins for which antibody development is difficult (Beekwilder et al. (1999) Gene 228:23-31; Pacheco et al. (2015) Amb Express, 5). Second, virus particles are less thermally and chemically labile than antibodies, dramatically simplifying the large-scale production, storage and transport of biosensors that rely on virus-based bioaffinity layers (Hayhurst et al, Curr. Opin. Chem. Biol. 5:683-689 (2001)). Third, virus particles that are capable of antibody-like affinities can be produced in quantity at lower costs (Weiss et al, Anal. Chem. 80:3082-3089 (2008)).

It has been demonstrated that engineered M13 phage could be immobilized by physisorption onto the gold transducer of an acoustic wave sensor (Petrenko et al, (2003) J. Microbiol. Meth. 53:253-262) and, somewhat later (Nanduri et al (2007) Biosens. Bioelectron. 22:986-992), to a gold quartz crystal microbalance electrode, enabling the detection in both cases of β-galactosidase (Petrenko et al, (2003) J. Microbiol. Meth. 53:253-262; Nanduri et al (2007) Biosens. Bioelectron. 22:986-992). Subsequently, in 2007 Cosnier et al. (Ionescu et al. (2007) Anal. Chem. 79:8662-8668) demonstrated biosensors based upon the virus T7 capable of detecting human antibodies to the West Nile virus.

New and improved biosensors are needed. There are provided herein, inter alia, solutions to these and other problems in the art.

### BRIEF SUMMARY

The disclosure provides electrochemical cells comprising: (a) a potentiostat electronically connecting a first electrode and a second electrode; (b) a first electronically conductive polymer between said first electrode and said second electrode; (c) a viral composition layer above said electronically conductive polymer, the viral composition layer comprising: a whole viral particle comprising a recombinant viral surface receptor; and a second electronically conductive polymer. In aspects, the electrochemical cell further comprises a cell layer forming a liquid-holding cell capable of a holding liquid; wherein the liquid-holding cell comprises a bottom portion comprising the first electrode and the second electrode. In aspects, the disclosure provides a diagnostic kit comprising the electrochemical cell and instructions for use. In aspects, the disclosure provides method of detecting a biomolecule in a sample by (i) contacting the first electrode and the second electrode of the electrochemical cell with a sample; and (ii) measuring the current of the sample, thereby detecting the biomolecule in the sample. In aspects, the current is measured by electrochemical impedance spectroscopy. In aspects, the sample is a biological sample.

The disclosure provides biosensors which comprise an electrochemical cell comprising: (a) a potentiostat electronically connecting a first electrode and a second electrode; (b) a first electronically conductive polymer between said first electrode and said second electrode; (c) a viral composition layer above said electronically conductive polymer, the viral composition layer comprising: a whole viral particle comprising a recombinant viral surface receptor; and a second electronically conductive polymer. In aspects, the biosensor further comprises a cell layer forming a liquid-holding cell capable of a holding liquid; wherein the liquid-holding cell comprises a bottom portion comprising the first electrode and the second electrode. In aspects, the biosensor further comprises a biological sample. In aspects, the disclosure provides a diagnostic kit comprising the biosensor and instructions for use. In aspects, the disclosure provides method of detecting a biomolecule in a sample by (i) contacting the biosensor with a sample; and (ii) measuring the current of the sample, thereby detecting the biomolecule in the sample. In aspects, the current is measured by electrochemical impedance spectroscopy. In aspects, the sample is a biological sample.

The disclosure provides methods of forming a biosensor with increased sensitivity, the method comprising modifying a biosensor by (i) decreasing the thickness of the first electronically conductive polymer, (ii) increasing the recombinant viral surface receptor copy number, or (iii) decreasing the thickness of the first electronically conductive polymer and increasing the recombinant viral surface receptor copy number; thereby forming the biosensor with increased sensitivity relative to the original biosensor. In aspects, the methods further comprise detecting a biomolecule in a sample using the biosensor.

The disclosure provides methods of forming a biosensor with decreased sensitivity, the method comprising modifying a biosensor by (i) increasing the thickness of the first electronically conductive polymer, (ii) decreasing the recombinant viral surface receptor copy number, or (iii) increasing the thickness of the first electronically conductive polymer and decreasing the recombinant viral surface receptor copy number; thereby forming a biosensor with decreased sensitivity relative to the original biosensor. In aspects, the methods further comprise detecting a biomolecule in a sample using the biosensor.

These and other embodiments and aspects of the disclosure are described in detail herein.

### BRIEF DESCRIPTION OF THE DRAWINGS

FIGS. 1A-1C show the Virus Bioresistor (VBR). FIG. 1A: Schematic diagram of a VBR showing critical components and dimensions. FIG. 1B: A buffered salt solution alters the solution resistance, Rₛₒₗₙ, but not the resistance of the VBR channel, R_{VBR}. FIG. 1C: In the presence of a target protein (HSA in this case), R_{VBR} is increased, enabling determination of its concentration.

FIGS. 2A-2E show VBR biosensor fabrication. FIG. 2A: Two pairs of gold-electrodes from which two VBRs are prepared. The gold electrodes have width of 2 mm and their separation of 1.5 mm defines the channel length of these devices. The two pairs of gold electrodes are separated by 0.5 mm. FIG. 2B: A layer of PEDOT:PSS is spin-coated onto the gold-electrode device and baked for 1 hr at 90° C. FIG. 2C: A 2 mm×2 mm PMMA cell is attached defining the area of the bioaffinity layer followed by incubation of PEDOT:PSS in PBS for 90 minutes. FIG. 2D: A virus-PEDOT top layer is electropolymerized on top of the PEDOT-PSS bottom layer by using ≈100 μL of plating solution and applying two oxidizing voltammetric scans. FIG. 2E: The virus-PEDOT plating solution is removed and the cell is rinsed. Electrodes are used to enable impedance measurements at each of the two VBR sensors. One background impedance measurement is acquired in buffer, and a second in a solution containing added HSA. The calculated ΔR_{VBR} is used to determine the HSA concentration in this sample with reference to a calibration curve.

FIGS. 3A-3H show electrodeposition and SEM/AFM characterization of virus-PEDOT bioaffinity layers. FIG. 3A: Electrodeposition of a virus-PEDOT film on a PEDOT-PSS film using cyclic voltammetry (50 mV/s). the virus-PEDOT top layer is prepared by two cycles from an aqueous virus-EDOT solution containing 2.5 mM EDOT, 12.5 mM LiClO₄, and 8 nM HSA phage. FIG. 3B: cross-sectional scanning electron microscopy (SEM) image of a PEDOT-PSS/virus-PEDOT film. The PEDOT-PSS bottom layer and virus-PEDOT top layer can be distinguished. FIG. 3C: Plan view SEM image of a solution containing 2.5 mM EDOT, 12.5 mM LiClO₄. FIG. 3D: Plan view SEM image of a virus-PEDOT film prepared as described in FIG. 3A. FIGS. 3E-3H: Atomic force microscopy (AFM) images of PEDOT films (FIGS. 3E and 3G) and virus-PEDOT films (FIGS. 3F and 3H). The same AFM image data are represented in two ways: FIGS. 3E and 3F show height versus position data while FIGS. 3G and 3H show a three-dimensional rendering of these the same data shows in FIGS. 3E and 3F. The rms roughness for PEDOT and virus-PEDOT films are ≈5 nm and ≈10 nm, respectively.

FIGS. 4A-4H show orthogonal measurement of Rₛₒₗₙ and R_{VBR} using a VBR biosensor. Nyquist plots summarizing the impedance response of VBRs from 1 Hz to 10 kHz with equivalent circuit fits (solid line traces). FIGS. 4A-4C show VBRs in solutions of run buffer of: FIG. 4A) 1×PBS, FIG. 4B) 2.5×PBS, FIG. 4C) 5×PBS, before and after exposure to 75 nM HSA in the same buffer. FIGS. 4D and 4E show plots of Rₛₒₗₙ and R_{VBR} as a function of buffer concentration extracted from the data of FIGS. 4A, 4B, and 4C. Shown are the values of these two circuit elements in pure buffer, and in buffer with added 75 nM HSA, as indicated. FIGS. 4F-4H show experiment in which the HSA concentration is increased from 0 nM (1×PBS) to 750 nM (in 1×PBS) showing the invariance of Rₛₒₗₙ and the linear increase in R_{VBR}.

FIGS. 5A-5C show calibration plots for 20 VBRs exposed to HSA concentrations 7.5 nM-900 nM generated by two methods (FIG. 6A) sensing signal ΔZᵣₑ, measured at 5 Hz, versus concentration, and (FIG. 6B) sensing signal defined as R_{VBR}, versus concentration.

FIGS. 6A-6B show VBR specificity and speed. FIG. 6A shows a specificity assay. Center bars represent three VBRs with PEDOT films containing HSA binding phage exposed to 750 nM HSA; Right bars show the response to a 750 nM BSA solution of three VBRs containing HSA binding phage; Left bars show the response to a 750 nM HSA solution for three VBRs containing STOP4 phage that have no affinity for HSA. FIG. 6B shows real time VBR sensing data. Responses for three VBR sensors are shown for [HSA] exposures of 220, 370, and 600 nM that show response times of 30 s, 3 s, and 3 s, respectively. The specificity assay summarized in FIG. 6A are also repeated here, in real-time sensing format, again showing no measurable responses.

FIG. 7 shows DL-1 phage and DJ-1 protein loaded into the PEDOT film of the sensor.

FIG. 8 shows the fabrication steps of VBRs for HSA.

FIG. 9 shows spin-coating of the base layer of baked PEDOT:PSS to yield a range of DC resistances across the electrodes.

FIG. 10 shows that as the base layer resistance is increased, the VBR signal increases by orders of magnitude.

FIG. 11 shows VBR signal for varying concentrations of DJ1 protein.

FIG. 12 shows VBR signal for no phage control, Stop4 control, and DL1 phage.

FIG. 13 shows a schematic representation of the phage-based sandwich-made bioresistor fabrication for high specificity DJ-1 detection.

FIG. 14 shows Nyquist diagrams for each immobilization step at carbon nanopowder electrode and phage/carbon nanopowder biosensor recorded in PBS, pH 7.4, in a solution of 10 nM DJ-1 and in a solution containing a second binding phage.

FIG. 15 shows a comparison of binding affinities between batches of the old (UCI lab prep B) and new batches with improved affinity (PT lab preps A-C) of HSA-binding filamentous M13 phage produced by us, assayed by direct ELISA.

FIG. 16 is the operation flow chart described in Example 3 for the propagation of M13 phage-displayed ligands from phagemids.

FIG. 17 provides the equivalent circuits and equations representing the electrical response of a VBR biosensor. ^{a} is the capacitive equivalent circuit. ^{b} is the equivalent circuit with constant phase elements (CPEs).

### DETAILED DESCRIPTION

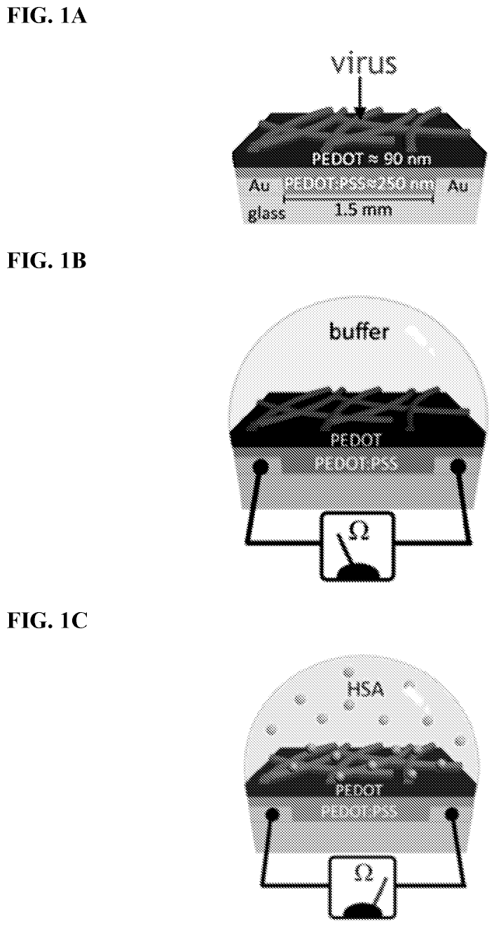

In embodiments, the virus bioresistor (or VBR), provides the means for incorporating thousands of virus particles into an electrical circuit (FIGS. 1A-1C). One element of the VBR is an electronically conductive channel composed of an electrically conductive polymer (e.g., poly(3,4-ethylenedioxythiophene) or PEDOT) into which virus particles (e.g., M13 virus particles) are embedded (FIG. 1A). Individual M13 virus particles may be filamentous with dimensions of 6 nm (width)×1.0 μm (length). The recognition and binding of target molecules to thousands of M13 virus particles embedded in this polymeric channel may be signaled by an electrical impedance signature that is measureable by an external circuit (FIG. 1B-1C).

The impedance response of the VBR may be modeled by a simple equivalent circuit containing just three circuit elements: a solution resistance (Rₛₒₗₙ), a channel resistance (R_{VBR}), and an interfacial capacitance (C_{VBR}). Information on target binding may be contained in the R_{VBR}, which can be measured either at a single frequency, or with higher precision from the best fit of the Nyquist plot across 40 or 50 discrete frequencies using this equivalent circuit.

Demonstrated herein, for example, is the VBR concept of using a model system in which human serum albumin (HSA, 66 kDa) is detected in a phosphate buffer solution. The VBRs may have a baseline dc resistance of 200-250Ω (either in air or in an aqueous buffer solution), and may be capable of producing large signals (ΔR_{VBR}≈250Ω, or ΔR_{VBR}/Rₒ≈100%) for the detection of HSA in phosphate buffer solutions across the entire HSA binding curve ranging from [HSA]=7.5 nM to 900 nM.

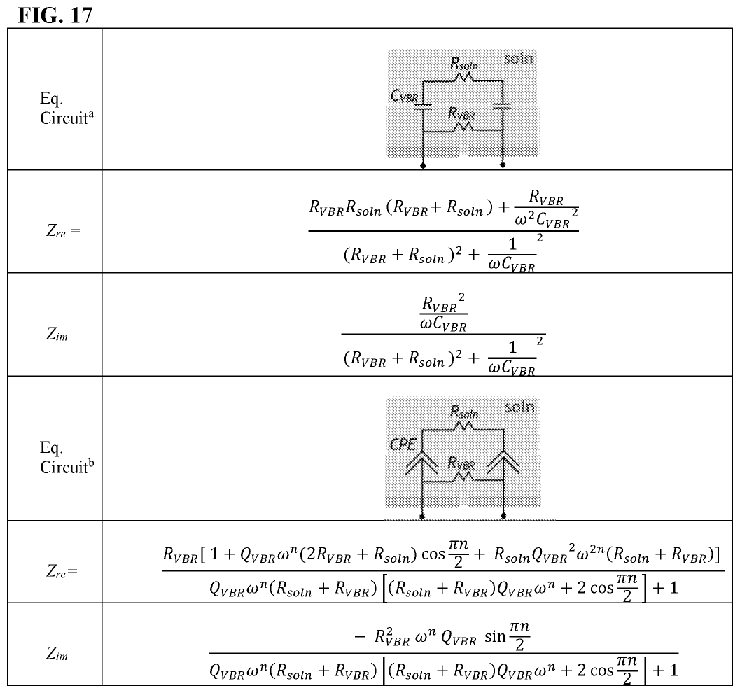

As shown in FIG. 17, analytical equations for the real and imaginary components of the complex impedance, Zᵣₑ and Zᵢₘ, may be used to fit experimental impedance data to extract the values of the three circuit elements: Rₛₒₗₙ, R_{VBR}, and C_{VBR}. A version of the equivalent circuit in which a constant phase element (CPE) may be substituted for each capacitor is used for this purpose because better agreement between calculated and experiment impedance data are obtained, resulting in improved precision for the measurement of R_{VBR} (FIG. 17). The impedance of a CPE, Z_{CPE}, and the capacitive impedance, Z_{C}, are defined by these equations:

$${Z}_{C} = \frac{1}{i ω C} {Z}_{C P E} = \frac{1}{i ω {Q}^{n}}$$

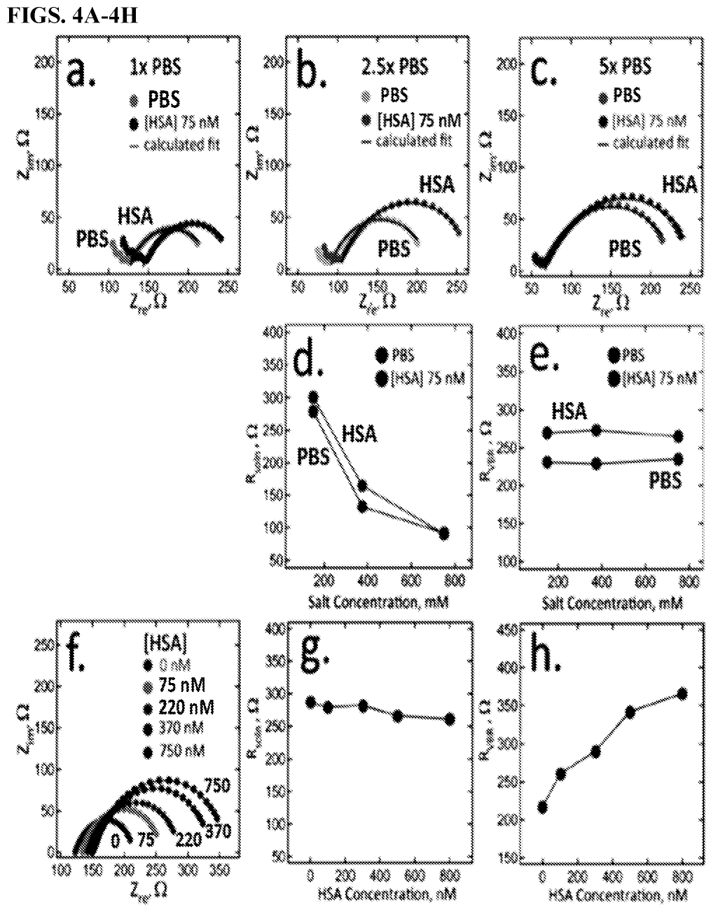

The VBR may produce a distinctive impedance response consisting of a semicircular Nyquist plot (Zim versus Zre as a function of frequency) (FIGS. 4A-4C). This response resembles the Randles equivalent circuit that is commonly seen for electrochemical biosensors operating in the presence of an added redox species, such as [Fe(CN)6]^{3-/4-}. The semicircular Nyquist plot for electro-chemical biosensors derives from electron transfer to/from the redox species present in the solution. When a redox species is not added, no semicircle is observed. The VBR produces a semicircular Nyquist plot without added redox species. This is because the VBR channel presents a parallel resistance (i.e., dominated by electron conduction through the polymer composite VBR) and capacitance (i.e., produced by the non-Faradaic charging and discharging of the electrical double layer at the surface of the VBR). The semicircular Nyquist plots aids in the precision with which RVBR can be measured (just as it does in electrochemical biosensors that use the diameter of this semicircle) the so-called charge transfer resistance to transduce target binding.

In spite of the fact that the electrical signal generated by VBRs derives purely from ensembles of biological entities, extremely high sensor-to-sensor reproducibility of this signal is attainable for the response of VBR biosensors culminating in a coefficient-of-variation of the measured [HSA] for 20 sensors less than 10% across the entire HSA binding curve. The VBR achieves these metrics using a two-terminal, monolithic device architecture that is simple, robust, manufacturable, and inexpensive. No reagents and no sandwich amplification of the impedance signal is required and no redox species are added to the test solution. Collectively, these data demonstrate that VBR will provide rapid and inexpensive urine and blood-based assays at the point-of-care.

VBR biosensors may be able to distinguish between changes in the electrical resistance of the test solution, caused by variations in the salt concentration for example, and the concentration of target molecules present in this solution. Information on the electrical conductivity of the solution is contained in Rₛₒₗₙ whereas the concentration of target protein is encoded by R_{VBR} and there is virtually no cross-talk in these two circuit elements. For example, Nyquist plots (Zᵢₘ versus Zᵣₑ as a function of frequency) for a VBR in three PBS solutions of 1×PBS, 2.5×PBS and 5×PBS show the same ΔR_{VBR}=R_{VBR,HSA}−R_{VBR,buffer} signal for 75 nM HSA (FIG. 4E) independent of the salt concentration over this entire range, even as Rₛₒₗₙ decreases dramatically with increasing salt (FIG. 4D).

VBR has the ability to parse changes in impedance due to solution resistance. The complimentary experiment is to vary [HSA] in a 1×PBS buffer solution (FIG. 4F). Here, Nyquist plots are shown for five buffer solutions containing [HSA]=0 nM, 70 nM, 220 nM, 370 nM, and 750 nM. In this case, a quasi-linear increase in ΔR_{VBR} with [HSA] is measured (FIG. 4H) while Rₛₒₗₙ remains constant (FIG. 4G). This property of VBRs (i.e., the ability to parse changes in impedance due to the solution resistance and target binding) provides an enormous advantage in terms of the application of this biosensor technology to bodily fluids where salt concentrations are unknown and uncontrolled.

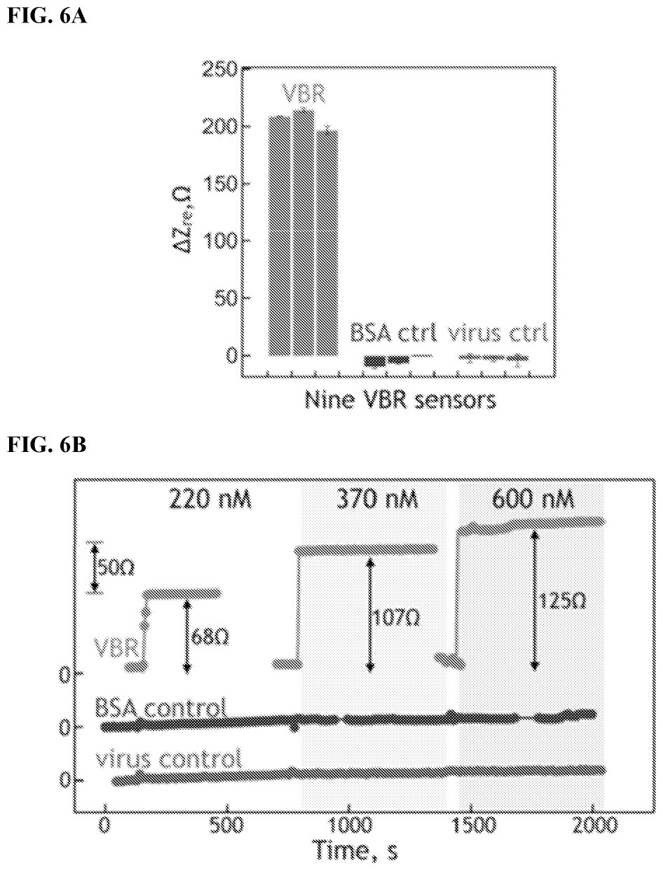

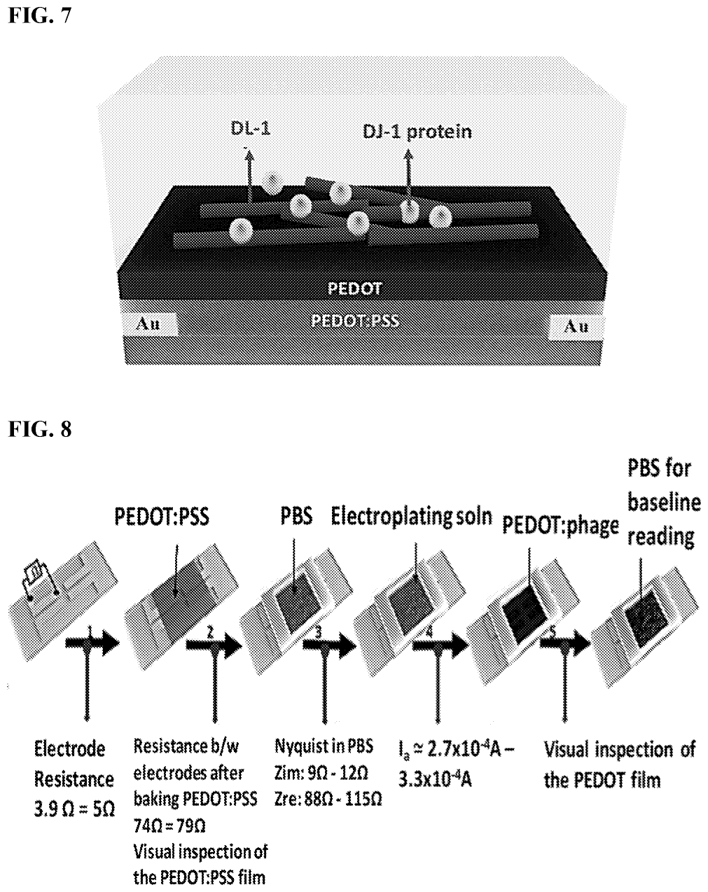

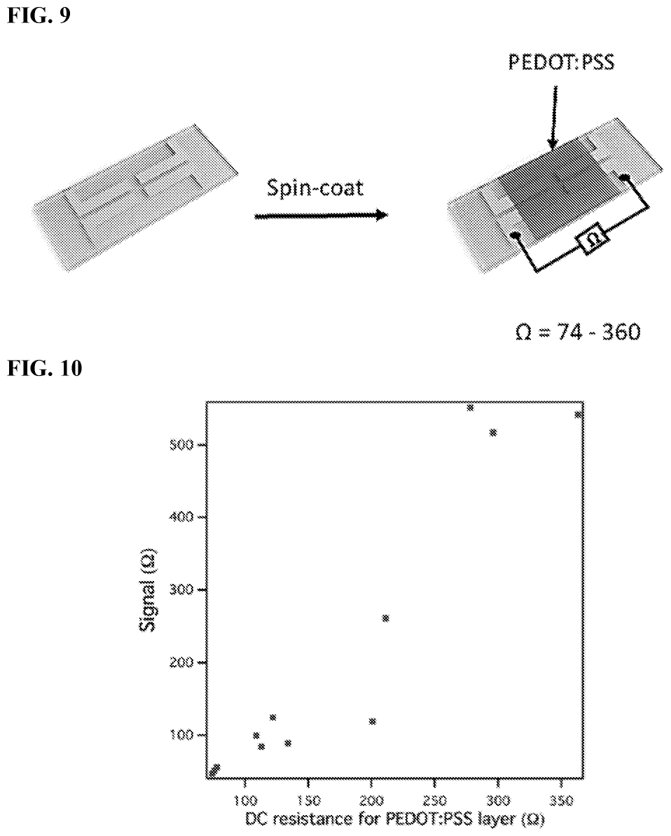

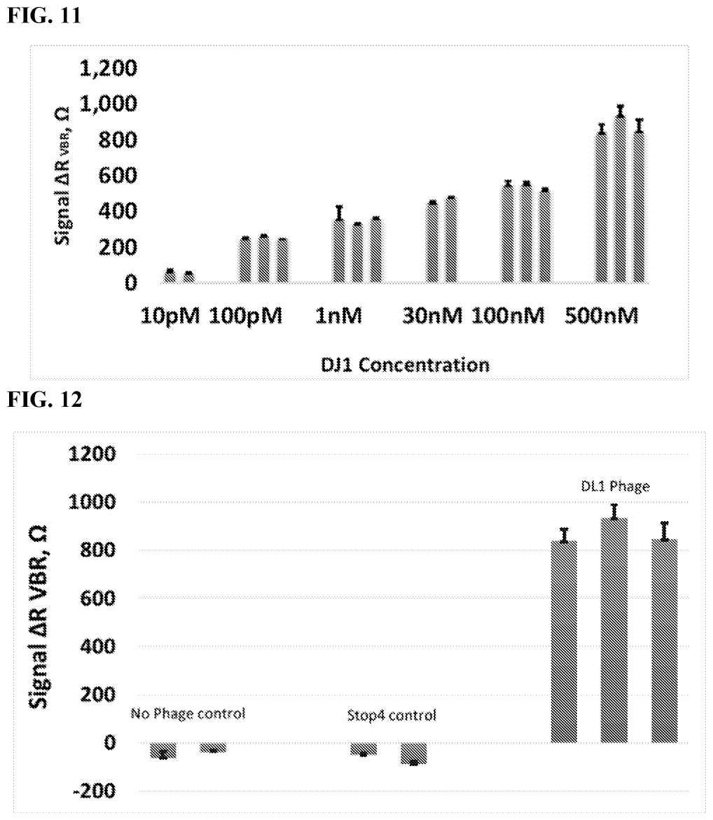

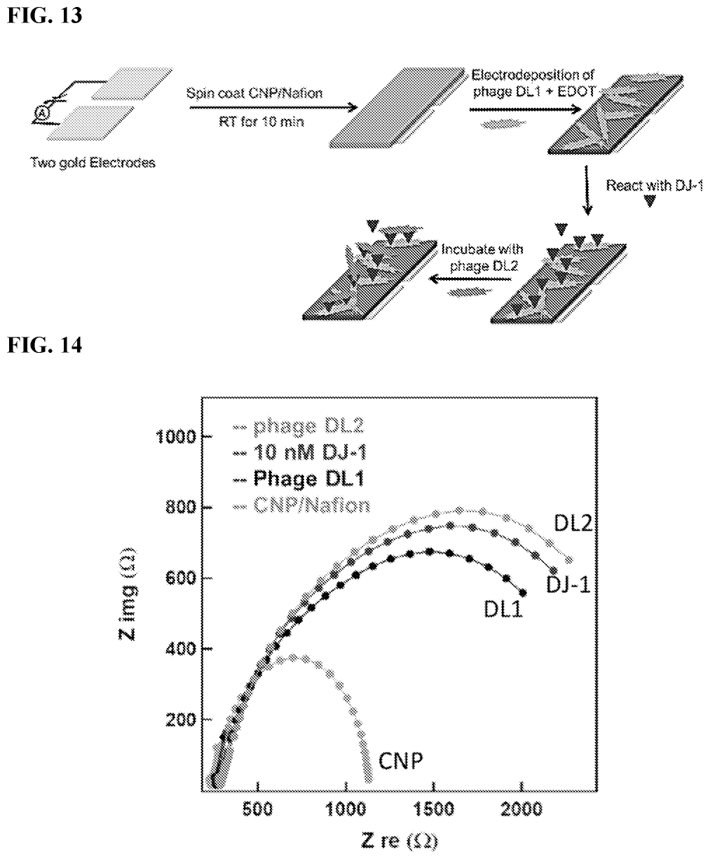

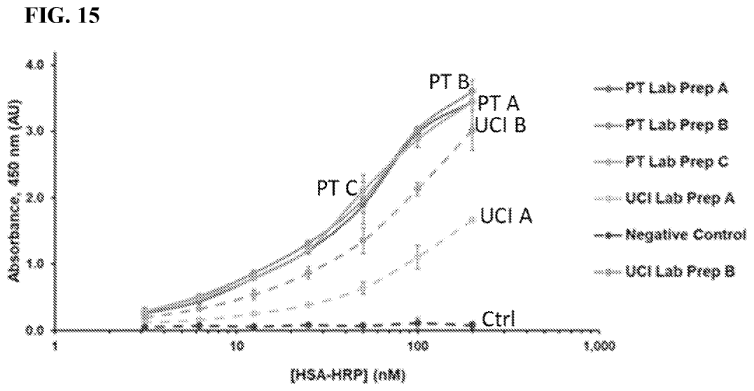

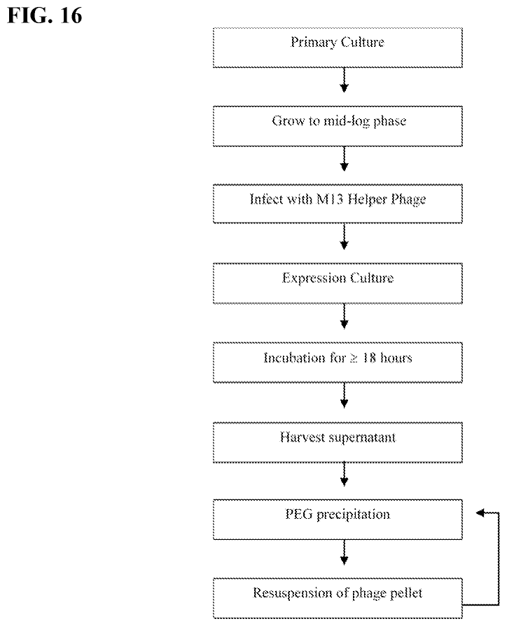

In addition to sensitivity and reproducibility, selectivity and speed are the two other attributes important for biosensors. Selectivity may be assessed by measuring the response of VBRs containing HSA-binding virus particles for bovine serum albumin, BSA, which is identical in size to HSA and has 70% amino acid homology (FIGS. 6A-6B). No measureable response is observed in these experiments. VBRs have also been prepared using wild-type virus particles that have no pendent polypeptides as required for specific binding of HSA. These devices show virtually no signal for HSA (FIGS. 6A-6B). Both control VBR biosensors show less than ˜1Ω in of change R_{channel} in comparison to ˜200Ω resistance increase for HSA-virus-PEDOT films against 750 nM HSA. The impedance response for VBRs gives excellent binding signal specific to HSA at 200× over background.

Real-time VBR measurements (FIG. 6B) allow the response time of these devices to be directly measured. A rapid (3-30 second) step-wise increase in ΔZᵣₑ followed by near instantaneous settling of Z_{Re} at the higher value (FIG. 6B) was observed. This constitutes a near ideal response function for a biosensor and demonstrates the utility of VBRs for point-of-care applications.

The virus particles can be engineered to bind different proteins which extends the scope of this two-terminal, monolithic device architecture that is simple, robust, manufacturable, and inexpensive. In aspects, no reagents and no sandwich amplification of the impedance signal is required and no redox species are added to the test solution. Data provided herein demonstrate, for example, the feasibility of adapting the VBR concept to rapid, inexpensive urine and blood-based assays at the point-of-care.

Definitions

The terms “biosensor,” “bioresistor,” “viral bioresistor,” “VBR biosensor,” or “VBR” refer to a device for detecting and measuring quantities or changes in a biochemical or chemical substance, in which a microelectronic component registers reactions related to the substance and translates them into data, or a device that detects, records, and transmits information regarding a physiological change or process, or a device that uses biological materials, such as enzymes, to monitor the presence of various chemicals in a substance. In aspects, the biosensor is a point of care (PoC) biosensor that comprises the electrochemical cells described herein.

The term “electrochemical cell” refers to a device having two electrodes connected by an electron conductor and spatially separated by an ionic conductor and that converts chemical energy into electrical energy or vice versa when a chemical reaction is occurring in the cell. In aspects, the electrochemical cell comprises a potentiostat electronically connected to a first electrode and a second electrode. In aspects, the electrochemical cell further comprises a first electronically conductive polymer between the first electrode and the second electrode. In aspects, the electrochemical cell further comprises a viral composition layer above the electronically conductive polymer, where the viral composition layer comprises a whole viral particle comprising a recombinant viral surface receptor; and a second electronically conductive polymer. In aspects, the electrode comprise a metal, a carbon, or a combination thereof. Exemplary metals for electrodes include gold, platinum, silver, palladium, rhodium, lead, copper, zinc, and combinations thereof.

The term “potentiostat” refers to a device to control or maintain the potential difference between electrodes (e.g., between a first electrode and a second electrode) at a constant level in an electrochemical cell.

“Electrically conductive polymer” refers to an organic polymer that conducts electricity. Examples of electrically conductive polymers include carbon polymers, polyfluorenes, polyphenylenes, polypyrenes, polyazulenes, polynaphthalenes, polypyrroles, polycarbazoles, polyindoles, polyazepines, polyanilines, polythiophenes, poly(3,4-ethylenedioxythiophene), poly(p-phenylene sulfide), polyacetylenes, poly(p-phenylene vinylene) and the like. Electrically conductive polymers can be modified with functional groups (e.g., hydroxy, sulfo) to impart desired properties to the polymer (e.g., water solubility). Such electrically conductive polymers modified with functional groups include poly(3,4-ethylenedioxythiophene) polystyrene sulfonate (PEDOT:PSS), and the like.

The term “carbon polymer” refers to a polymer prepared using carbon nanopowder (non-graphitic carbon). For example, carbon nanopoweder can be prepared by a process comprising the steps of (a) preparing a composition comprising a carbon (e.g., about 250 mg carbon nanopowder having less than 100 nm particle size nanopowder in 1.5 mL of NAFION® 117 in a 5% mixture of lower aliphatic alcohols and water); (b) vortexing and sonicating the composition comprising the carbon nanopowder (e.g., at room temperature for about 30 minutes); and (c) spinning and coating the composition comprising the carbon nanopowder on an electrode (e.g., gold electrode). NAFION® 117 (DuPont) is a non-reinforced film based on a chemically stabilized perfluorosulfonic acid/polytetrafluoroethylene copolymer in the acid (H+) form.

The terms “virus” or “virus particle” or “whole viral particle” are used according to its plain ordinary meaning within virology and refer to a virion including the viral genome (e.g. DNA, RNA, single strand, double strand), viral capsid and associated proteins, and in the case of enveloped viruses (e.g. herpesvirus), an envelope including lipids and optionally components of host cell membranes, and/or viral proteins.

The term “viral composition layer” refers to a composition comprising: (i) a whole viral particle which comprises a recombinant viral surface receptor, and (ii) an electronically conductive polymer.

The term “recombinant viral surface receptor” refers to a protein (e.g. receptor) that is expressed on the surface of the whole viral particle and that is capable of binding a complementary ligand (e.g., a ligand protein). In embodiments, the recombinant viral surface receptor is expressed from a recombinant nucleotide sequence comprising an inducible promoter. In embodiments, the recombinant viral surface receptor is capable of binding to a cell surface marker (e.g., a cancer cell surface marker).

The term “ligand” refers to a composition (e.g., atom, molecule, ion, molecular ion, compound, particle, protein, peptide, nucleic acid, oligosaccharide, polysaccharide, or small molecule) capable of binding (e.g. specifically binding) to a protein (e.g. receptor, such as a recombinant viral surface receptor) to form a complex. A ligand as provided herein may without limitation be a biomolecule (e.g., hormones, cytokines, proteins, nucleic acids, lipids, carbohydrates, cellular membrane antigens and receptors (neural, hormonal, nutrient, and cell surface receptors or their ligands)); whole cells or lysates thereof (e.g., prokaryotic (e.g., pathogenic bacteria), eukaryotic cells (e.g., mammalian tumor cells); viruses (e.g., retroviruses, herpesviruses, adenoviruses, lentiviruses and spores); chemicals (e.g., solvents, polymers, organic materials, small molecules); therapeutic molecules (e.g., therapeutic drugs, abused drugs, antibiotics); environmental pollutants (e.g., pesticides, insecticides, toxins). In aspects, the ligand is a cell surface marker binding moiety (i.e., a composition that recognizes and binds to a cell surface marker).

The term “cell surface marker” refers to composition (e.g., atom, molecule, ion, molecular ion, compound, particle, protein, peptide, nucleic acid, oligosaccharide, polysaccharide, or small molecule) found on the external cell wall or plasma membrane of a specific cell type or a limited number of cell types (Molday et al, Histochemical Journal 12:273-315 (1980); Hewett, International Journal of Biochemistry & Cell Biology 33:325-335 (2001); Pembrey et al., Applied and Environmental Microbiology 65:2877-2894 (1999)).

The terms “specific binding” or “specifically binds” refer to two molecules forming a complex that is relatively stable under physiologic conditions.

Methods for determining whether a ligand binds to a protein (e.g. receptor) and/or the affinity for a ligand to a protein are known in the art. For example, the binding of a ligand to a protein can be detected and/or quantified using a variety of techniques such as, but not limited to, Western blot, dot blot, surface plasmon resonance method (e.g., BIAcore system; Pharmacia Biosensor AB, Uppsala, Sweden and Piscataway, N.J.), isothermal titration calorimetry (ITC), or enzyme-linked immunosorbent assays (ELISA). Immunoassays which can be used to analyze immunospecific binding and cross-reactivity of the ligand include, but are not limited to, competitive and non-competitive assay systems using techniques such as Western blots, RIA, ELISA (enzyme linked immunosorbent assay), “sandwich” immunoassays, immunoprecipitation assays, immunodiffusion assays, agglutination assays, complement-fixation assays, immunoradiometric assays, and fluorescent immunoassays. Such assays are routine and well known in the art.

“Electrochemical impedance spectroscopy” refers to a method of measuring the electrical impedance of a substance as a function of the frequency of an applied electrical current in an electrochemical cell.

The terms “gap” or “space” refer to a distance between electrodes that allows for the passage or flow of a voltage or current between the electrodes that can be measured by, for example, electrochemical impedance spectroscopy.

The term “cell layer” refers to a device comprising a liquid-holding cell, a first electrode, and a second electrode. In aspects, the cell layer comprises a polymer. In aspects, the cell layer comprises an acrylic polymer or an acrylic copolymer. In aspects, the cell layer is adjacent a solid support.

The term “liquid-holding cell” refers to a compartment, a cavity, a hollow, or a unit in a device receiving a volume of a liquid sample (e.g., biological sample). In aspects, the liquid-holding cell is a flow cell that comprises an inlet port and an outlet port that allows the sample (e.g., biological sample) to flow through the device. In aspects, the liquid-holding cell further comprises a portion (e.g., bottom portion) that includes the first electrode and the second electrode.

“Acrylic polymer” refers to polymers comprised of acrylate monomers, e.g., homopolymers of acrylic acid crosslinked with allyl ether pentaerythritol, allether of sucrose, or allyl ether of propylene. Exemplary acrylic monomers include acrylic acid, methacrylate (methacrylic acid), methyl acrylate, ethyl acrylate, butyl acrylate, 2-chloroethyl vinyl ether, 2-ethylhexyl acrylate, hydroxyethyl methacrylate, methyl methacrylate, ethyl methacrylate, butyl methacrylate, and the like. Acrylic polymers are commercially available in varying molecular weights, such as from about 2,000 Daltons to about 1,500,000 Daltons.

“Acrylic copolymer” refers to polymers comprised of at least two different acrylate monomers. Exemplary acrylic monomers include acrylic acid, methacrylate (methacrylic acid), methyl acrylate, ethyl acrylate, butyl acrylate, 2-chloroethyl vinyl ether, 2-ethylhexyl acrylate, hydroxyethyl methacrylate, methyl methacrylate, ethyl methacrylate, butyl methacrylate, and the like. Exemplary acrylic copolymers include copolymers of methacrylic acid and ethyl acrylate, and copolymer of methacrylic acid and Methyl methacrylate. Acrylic copolymers are commercially available.

The term “biomolecule” refers to a molecule that is made or naturally occurs in a living organism, such as amino acids, sugars, nucleic acids, proteins, polysaccharides, DNA and RNA. In embodiments, the biomolecules are hormones, cytokines, proteins, nucleic acids, lipids, carbohydrates, cellular membrane antigens and receptors (neural, hormonal, nutrient, and cell surface receptors) or their ligands. In aspects, the biomolecules are cancer cell markers. In aspects, the biomolecule is human serum albumin.

“Biological sample” refers to materials obtained from or derived from a subject or patient. A biological sample includes sections of tissues such as biopsy and autopsy samples, and frozen sections taken for histological purposes. Such samples include bodily fluids such as blood and blood fractions or products (e.g., serum, plasma, platelets, red blood cells, white blood cells, and the like), sputum, tissue, cultured cells (e.g., primary cultures, explants, and transformed cells), stool, urine, cerebral spinal fluid, lacrimal fluid, nipple aspirate fluid, synovial fluid, joint tissue, synovial tissue, synoviocytes, fibroblast-like synoviocytes, macrophage-like synoviocytes, immune cells, hematopoietic cells, fibroblasts, macrophages, T cells, etc. A biological sample is typically obtained from a eukaryotic organism, such as a mammal such as a primate e.g., chimpanzee or human; cow; dog; cat; a rodent, e.g., guinea pig, rat, mouse; rabbit; or a bird; reptile; or fish.

A “solid support” as provided herein refers to any material that can be modified to contain discrete individual sites for the attachment or association of an electronically conductive polymer as provided herein, and that is amenable to the methods provided herein. Examples of solid supports include without limitation, glass and modified or functionalized glass (e.g., carboxymethyldextran functionalized glass), plastics (including acrylics, polystyrene and copolymers of styrene and other materials, polypropylene, polyethylene, polybutylene, polyurethanes, polytetrafluoroethylene, TEFLON® (The Chemours Co.), etc.), polysaccharides, nylon or nitrocellulose, composite materials, ceramics, and plastic resins, silica or silica-based materials including silicon and modified silicon (e.g., patterned silicon), carbon, metals, quartz (e.g., patterned quartz), inorganic glasses, plastics, optical fiber bundles, and a variety of other polymers (e.g., electronically conductive polymers such as poly-3,4-ethylenedioxythiophene, PEDOT). In general, the solid support allows optical detection and does not appreciably fluoresce. The solid support may be planar (e.g., flat planar substrates such as glass, polystyrene and other plastics and acrylics). Although it will be appreciated by a person of ordinary skill in the art that other configurations of solid supports may be used as well; for example, three dimensional configurations can be used. The solid support may be modified to contain discrete, individual sites (also referred to herein as “wells”) for polymer binding. These sites generally include physically altered sites, i.e. physical configurations such as wells or small depressions in the substrate that can retain the polymers. The wells may be formed using a variety of techniques well known in the art, including, but not limited to, photolithography, stamping techniques, molding techniques and microetching techniques. It will be appreciated by a person of ordinary skill in the art that the technique used will depend on the composition and shape of the solid support. In aspects, physical alterations are made in a surface of the solid support to produce wells. In aspects, the solid support is a microtiter plate. In aspects, the solid support is glass. In aspects, the solid support is non-electronically conductive material.

“Nucleic acid” refers to deoxyribonucleotides or ribonucleotides and polymers thereof in either single-, double- or multiple-stranded form, or complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acids can be linear or branched. For example, nucleic acids can be a linear chain of nucleotides or the nucleic acids can be branched, e.g., such that the nucleic acids comprise one or more arms or branches of nucleotides. Optionally, the branched nucleic acids are repetitively branched to form higher ordered structures such as dendrimers and the like. Nucleic acids, including nucleic acids with a phosphothioate backbone can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides.

As used herein, the term “about” means a range of values including the specified value, which a person of ordinary skill in the art would consider reasonably similar to the specified value. In aspects, the term “about” means within a standard deviation using measurements generally acceptable in the art. In aspects, about means a range extending to +/−10% of the specified value. In aspects, about means the specified value.

The terms “polypeptide,” “peptide” and “protein” are used interchangeably herein to refer to a polymer of amino acid residues, wherein the polymer may be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymers. The terms apply to macrocyclic peptides, peptides that have been modified with non-peptide functionality, peptidomimetics, polyamides, and macrolactams.

A polypeptide, or a cell is “recombinant” when it is artificial or engineered, or derived from or contains an artificial or engineered protein or nucleic acid (e.g. non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

A “control” sample or value refers to a sample that serves as a reference, usually a known reference, for comparison to a test sample. For example, a test sample can be taken from a test condition, e.g., in the presence of a test compound, and compared to samples from known conditions, e.g., in the absence of the test compound (negative control), or in the presence of a known compound (positive control). A control can also represent an average value gathered from a number of tests or results. One of skill in the art will recognize that controls can be designed for assessment of any number of parameters. For example, a control can be devised to compare therapeutic benefit based on pharmacological data (e.g., half-life) or therapeutic measures (e.g., comparison of side effects). One of skill in the art will understand which controls are most appropriate in a given situation and be able to analyze data based on comparisons to control values. Controls are also valuable for determining the significance (e.g. statistical significance) of data. For example, if values for a given parameter are widely variant in controls, variation in test samples will not be considered as significant.

The term “diagnosis” refers to a relative probability that a disease (e.g. cancer, urinary tract infection, infection, or other disease) is present in the subject. Similarly, the term “prognosis” refers to a relative probability that a certain future outcome may occur in the subject with respect to a disease state. For example, in the context of the present invention, prognosis can refer to the likelihood that an individual will develop a disease (e.g. cancer, urinary tract infection, infection, or other disease), or the likely severity of the disease (e.g., duration of disease). The terms are not intended to be absolute, as will be appreciated by any one of skill in the field of medical diagnostics.

As used herein, a “diagnostically effective amount” of a composition described herein is an amount sufficient to produce a clinically useful characterization or measurement of a disease state, such as an infection or cancer, (e.g. in an individual, patient, human, mammal, clinical sample, tissue, biopsy). A clinically useful characterization or measurement of a disease state, such as an infection or cancer, (e.g. in an individual, patient, human, mammal, clinical sample, tissue, biopsy) is one containing sufficient detail to enable an experienced clinician to assess the degree and/or extent of disease for purposes of diagnosis, monitoring the efficacy of a therapeutic intervention, and the like.

“Subject,” “patient,” “subject in need thereof,” “patient in need thereof,” and the like refer to a living organism. Non-limiting examples include humans, other mammals, bovines, rats, mice, dogs, monkeys, goat, sheep, cows, deer, and other non-mammalian animals. In aspects, a subject is human.

The disclosure provides electrochemical cells comprising: (a) a potentiostat electronically connecting a first electrode and a second electrode; (b) a first electronically conductive polymer between the first electrode and the second electrode; and (c) a viral composition layer above the electronically conductive polymer, wherein the viral composition layer comprises (i) a whole viral particle comprising a recombinant viral surface receptor; and (ii) a second electronically conductive polymer. In aspects, the disclosure provides diagnostic kits comprising the electrochemical cell and instructions for use.

The disclosure provides biosensors, where the biosensors comprise electrochemical cells comprising: (a) a potentiostat electronically connecting a first electrode and a second electrode; (b) a first electronically conductive polymer between the first electrode and the second electrode; and (c) a viral composition layer above the electronically conductive polymer, wherein the viral composition layer comprises (i) a whole viral particle comprising a recombinant viral surface receptor; and (ii) a second electronically conductive polymer. In aspects, the disclosure provides diagnostic kits comprising the biosensor and instructions for use.

In embodiments, the first electrode and the second electrode comprise a metal, carbon, or a combination thereof. In aspects, the first electrode and the second electrode comprise carbon. In aspects, the first electrode and the second electrode comprise a metal. In aspects, the first electrode and the second electrode each independently comprise gold, platinum, silver, palladium, rhodium, lead, copper, zinc, or a combination of two or more thereof. In aspects, the first electrode and the second electrode each independently comprise gold, platinum, silver, palladium, rhodium, lead, copper, or zinc. In aspects, the first electrode and the second electrode are different. In aspects, the first electrode and the second electrode are the same. In aspects, the first electrode and the second electrode comprise gold. In aspects, the first electrode and the second electrode comprise platinum. In aspects, the first electrode and the second electrode comprise silver. In aspects, the first electrode and the second electrode comprise palladium. In aspects, the first electrode and the second electrode comprise rhodium. In aspects, the first electrode and the second electrode comprise lead. In aspects, the first electrode and the second electrode comprise copper. In aspects, the first electrode and the second electrode comprise zinc.

In embodiments, the first electrode and the second electrode are separated by a space. In aspects, the first electrode and the second electrode are separated by a space from about 0.1 millimeter to about 5 millimeters. In aspects, the first electrode and the second electrode are separated by a space from about 0.5 millimeters to about 2.5 millimeters. In aspects, the first electrode and the second electrode are separated by a space from about 1.0 millimeter to about 2.0 millimeters. In aspects, the first electrode and the second electrode are separated by a space from about 1.1 millimeters to about 1.9 millimeters. In aspects, the first electrode and the second electrode are separated by a space from about 1.2 millimeters to about 1.8 millimeters. In aspects, the first electrode and the second electrode are separated by a space from about 1.3 millimeters to about 1.7 millimeters. In aspects, the first electrode and the second electrode are separated by a space from about 1.4 millimeters to about 1.6 millimeters. In aspects, the first electrode and the second electrode are separated by a space of about 0.5 millimeters. In aspects, the first electrode and the second electrode are separated by a space of about 0.6 millimeters. In aspects, the first electrode and the second electrode are separated by a space of about 0.7 millimeters. In aspects, the first electrode and the second electrode are separated by a space of about 0.8 millimeters. In aspects, the first electrode and the second electrode are separated by a space of about 0.9 millimeters. In aspects, the first electrode and the second electrode are separated by a space of about 1.0 millimeter. In aspects, the first electrode and the second electrode are separated by a space of about 1.1 millimeters. In aspects, the first electrode and the second electrode are separated by a space of about 1.2 millimeters. In aspects, the first electrode and the second electrode are separated by a space of about 1.3 millimeters. In aspects, the first electrode and the second electrode are separated by a space of about 1.4 millimeters. In aspects, the first electrode and the second electrode are separated by a space of about 1.5 millimeters. In aspects, the first electrode and the second electrode are separated by a space of about 1.6 millimeters. In aspects, the first electrode and the second electrode are separated by a space of about 1.7 millimeters. In aspects, the first electrode and the second electrode are separated by a space of about 1.8 millimeters. In aspects, the first electrode and the second electrode are separated by a space of about 1.9 millimeters. In aspects, the first electrode and the second electrode are separated by a space of about 2.0 millimeters. In aspects, the first electrode and the second electrode are separated by a space of about 2.1 millimeters. In aspects, the first electrode and the second electrode are separated by a space of about 2.2 millimeters. In aspects, the first electrode and the second electrode are separated by a space of about 2.3 millimeters. In aspects, the first electrode and the second electrode are separated by a space of about 2.4 millimeters. In aspects, the first electrode and the second electrode are separated by a space of about 2.5 millimeters.

In embodiments, the first electronically conductive polymer is a carbon polymer, a polyfluorene, a polyphenylene, a polypyrene, a polyazulene, a polynaphthalene, a polypyrroles, a polycarbazole, a polyindole, a polyazepine, a polyaniline, a polythiophene, a poly(3,4-ethylenedioxythiophene), a poly(p-phenylene sulfide), a polyacetylene, a poly(p-phenylene vinylene), or a combination of two or more thereof. In aspects, the first electrically conductive polymer is modified with a functional groups. In aspects, the functional group is a sulfonate moiety. In aspects, the functional group is a hydro moiety. In aspects, the first electronically conductive polymer comprises poly(3,4-ethylenedioxythiophene) polystyrene sulfonate (PEDOT:PSS). In aspects, the first electronically conductive polymer comprises a carbon polymer. In aspects, the first electronically conductive polymer is applied by spin coating.

In embodiments, the first electronically conductive polymer has a resistance from about 0.1 kOhm to about 5 kOhm. In aspects, the first electronically conductive polymer has a resistance from about 0.1 kOhm to about 3 kOhm. In aspects, the first electronically conductive polymer has a resistance from about 0.2 kOhm to about 2.8 kOhm. In aspects, the first electronically conductive polymer has a resistance from about 0.3 kOhm to about 2.7 kOhm. In aspects, the first electronically conductive polymer has a resistance from about 0.4 kOhm to about 2.6 kOhm. In aspects, the first electronically conductive polymer has a resistance from about 0.5 kOhm to about 3 kOhm. In aspects, the first electronically conductive polymer has a resistance from about 0.5 kOhm to about 2.5 kOhm. In aspects, the first electronically conductive polymer has a resistance from about 0.6 kOhm to about 2.4 kOhm. In aspects, the first electronically conductive polymer has a resistance from about 0.7 kOhm to about 2.3 kOhm. In aspects, the first electronically conductive polymer has a resistance from about 0.8 kOhm to about 2.2 kOhm. In aspects, the first electronically conductive polymer has a resistance from about 1 kOhm to about 2.5 kOhm. In aspects, the first electronically conductive polymer has a resistance from about 1 kOhm to about 2 kOhm.

In embodiments, the first electrically conductive polymer is present in a layer having a thickness from about 1 nm to about 1,000 nm. In aspects, the first electrically conductive polymer has a thickness from about 10 nm to about 500 nm. In aspects, the first electrically conductive polymer has a thickness from about 50 nm to about 450 nm. In aspects, the first electrically conductive polymer has a thickness from about 100 nm to about 400 nm. In aspects, the first electrically conductive polymer has a thickness from about 150 nm to about 350 nm. In aspects, the first electrically conductive polymer has a thickness from about 160 nm to about 340 nm. In aspects, the first electrically conductive polymer has a thickness from about 170 nm to about 330 nm. In aspects, the first electrically conductive polymer has a thickness from about 175 nm to about 325 nm. In aspects, the first electrically conductive polymer has a thickness from about 180 nm to about 320 nm. In aspects, the first electrically conductive polymer has a thickness from about 190 nm to about 310 nm. In aspects, the first electrically conductive polymer has a thickness from about 200 nm to about 300 nm. In aspects, the first electrically conductive polymer has a thickness from about 210 nm to about 290 nm. In aspects, the first electrically conductive polymer has a thickness from about 220 nm to about 280 nm. In aspects, the first electrically conductive polymer has a thickness from about 225 nm to about 275 nm. In aspects, the first electrically conductive polymer has a thickness from about 230 nm to about 270 nm. In aspects, the first electrically conductive polymer has a thickness from about 240 nm to about 260 nm. In aspects, the first electrically conductive polymer has a thickness from about 240 nm to about 250 nm. In aspects, the first electrically conductive polymer has a thickness from about 250 nm to about 260 nm. In aspects, the first electrically conductive polymer has a thickness from about 245 nm to about 255 nm. In aspects, the first electrically conductive polymer has a thickness of about 200 nm. In aspects, the first electrically conductive polymer has a thickness of about 210 nm. In aspects, the first electrically conductive polymer has a thickness of about 220 nm. In aspects, the first electrically conductive polymer has a thickness of about 225 nm. In aspects, the first electrically conductive polymer has a thickness of about 230 nm. In aspects, the first electrically conductive polymer has a thickness of about 240 nm. In aspects, the first electrically conductive polymer has a thickness of about 245 nm. In aspects, the first electrically conductive polymer has a thickness of about 250 nm. In aspects, the first electrically conductive polymer has a thickness of about 255 nm. In aspects, the first electrically conductive polymer has a thickness of about 260 nm. In aspects, the first electrically conductive polymer has a thickness of about 270 nm. In aspects, the first electrically conductive polymer has a thickness of about 275 nm. In aspects, the first electrically conductive polymer has a thickness of about 280 nm. In aspects, the first electrically conductive polymer has a thickness of about 290 nm. In aspects, the first electrically conductive polymer has a thickness of about 300 nm. An exemplary thickness of the first electrically conductive polymer is shown in FIG. 1A.

In embodiments, the electrochemical cell comprises a viral composition layer. In aspects, the viral composition layer is above the first electronically conductive polymer. In aspects, the viral composition layer comprises a whole viral particle and a second electronically conductive polymer. In aspects, the whole viral particle comprises a recombinant viral surface receptor. In aspects, the viral composition layer is above the first electrode and the second electrode. In aspects, the viral composition layer is adjacent to the first electrode and the second electrode. In aspects, the viral composition layer is above and adjacent to the first electrode and the second electrode. In aspects, the viral composition layer is applied by electrodeposition.

In embodiments, the second electronically conductive polymer is a carbon polymer, a polyfluorene, a polyphenylene, a polypyrene, a polyazulene, a polynaphthalene, a polypyrroles, a polycarbazole, a polyindole, a polyazepine, a polyaniline, a polythiophene, a poly(3,4-ethylenedioxythiophene), a poly(p-phenylene sulfide), a polyacetylene, a poly(p-phenylene vinylene), or a combination of two or more thereof. In aspects, the second electrically conductive polymer is modified with a functional groups. In aspects, the functional group is a sulfonate moiety. In aspects, the functional group is a hydro moiety. In aspects, the second electronically conductive polymer comprises poly(3,4-ethylenedioxythiophene) polystyrene sulfonate. In aspects, the second electronically conductive polymer comprises a carbon polymer. In aspects, the second electronically conductive polymer comprises poly(3,4-ethylenedioxythiophene). In aspects, the first electrically conductive polymer and the second electrically conductive polymer comprise the same polymer. In aspects, the first electrically conductive polymer and the second electrically conductive polymer comprise different polymers. In aspects, the first electronically conductive polymer comprises poly(3,4-ethylenedioxythiophene) polystyrene sulfonate, and the second electronically conductive polymer comprises poly(3,4-ethylenedioxythiophene). In aspects, the first electronically conductive polymer comprises a carbon polymer, and the second electronically conductive polymer comprises poly(3,4-ethylenedioxythiophene).

In embodiments, the viral composition layer has a thickness from about 1 nm to about 500 nm. In aspects, the viral composition layer has a thickness from about 10 nm to about 250 nm. In aspects, the viral composition layer has a thickness from about 10 nm to about 200 nm. In aspects, the viral composition layer has a thickness from about 30 nm to about 150 nm. In aspects, the viral composition layer has a thickness from about 40 nm to about 140 nm. In aspects, the viral composition layer has a thickness from about 50 nm to about 130 nm. In aspects, the viral composition layer has a thickness from about 55 nm to about 125 nm. In aspects, the viral composition layer has a thickness from about 60 nm to about 120 nm. In aspects, the viral composition layer has a thickness from about 65 nm to about 115 nm. In aspects, the viral composition layer has a thickness from about 70 nm to about 110 nm. In aspects, the viral composition layer has a thickness from about 75 nm to about 105 nm. In aspects, the viral composition layer has a thickness from about 80 nm to about 100 nm. In aspects, the viral composition layer has a thickness from about 85 nm to about 95 nm. In aspects, the viral composition layer has a thickness of about 70 nm. In aspects, the viral composition layer has a thickness of about 75 nm. In aspects, the viral composition layer has a thickness of about 80 nm. In aspects, the viral composition layer has a thickness of about 85 nm. In aspects, the viral composition layer has a thickness of about 90 nm. In aspects, the viral composition layer has a thickness of about 95 nm. In aspects, the viral composition layer has a thickness of about 100 nm. In aspects, the viral composition layer has a thickness of about 105 nm. In aspects, the viral composition layer has a thickness of about 110 nm.

In embodiments, the viral composition layer comprises a whole viral particle embedded within the second electronically conductive polymer. In aspects, the viral composition layer comprises a plurality of whole viral particles embedded within the second electronically conductive polymer. In aspects, the whole viral particle is a M13 virus particle. In aspects, the whole viral particle is a M13 filamentous virus particle. In aspects, the viral composition layer has an RMS surface roughness greater than 5 nm. In aspects, the viral composition layer has an RMS surface roughness from about 5 nm to about 25 nm. In aspects, the viral composition layer has an RMS surface roughness from about 5.5 nm to about 25 nm. In aspects, the viral composition layer has an RMS surface roughness from about 6 nm to about 25 nm. In aspects, the viral composition layer has an RMS surface roughness from about 6 nm to about 20 nm. In aspects, the viral composition layer has an RMS surface roughness from about 6 nm to about 15 nm. In aspects, the viral composition layer has an RMS surface roughness from about 9 nm to about 11 nm. In aspects, the viral composition layer has an RMS surface roughness from about 8 nm to about 12 nm. In aspects, the viral composition layer has an RMS surface roughness from about 7 nm to about 13 nm. In aspects, the viral composition layer has an RMS surface roughness from about 6 nm to about 14 nm. In aspects, the viral composition layers has an RMS surface roughness of about 6 nm. In aspects, the viral composition layers has an RMS surface roughness of about 7 nm. In aspects, the viral composition layers has an RMS surface roughness of about 8 nm. In aspects, the viral composition layers has an RMS surface roughness of about 9 nm. In aspects, the viral composition layers has an RMS surface roughness of about 10 nm. In aspects, the viral composition layers has an RMS surface roughness of about 11 nm. In aspects, the viral composition layers has an RMS surface roughness of about 12 nm. In aspects, the viral composition layers has an RMS surface roughness of about 13 nm. In aspects, the viral composition layers has an RMS surface roughness of about 14nm. In aspects, the viral composition layers has an RMS surface roughness of about 15 nm.

In aspects, the recombinant viral surface receptor is expressed from a recombinant nucleotide sequence comprising an inducible promoter. In aspects, the recombinant viral surface receptor is capable of binding to a cell surface marker. In aspects, the recombinant viral surface receptor is capable of binding to a cancer cell surface marker. In aspects, the recombinant viral surface receptor is capable of binding to a hormone, cytokine, protein, nucleic acid, lipid or carbohydrate. In aspects, the recombinant viral surface receptor is capable of binding to a hormone. In aspects, the recombinant viral surface receptor is capable of binding to a cytokine. In aspects, the recombinant viral surface receptor is capable of binding to a protein. In aspects, the recombinant viral surface receptor is capable of binding to a nucleic acid. In aspects, the recombinant viral surface receptor is capable of binding to a lipid. In aspects, the recombinant viral surface receptor is capable of binding to a carbohydrate.

In embodiments, the electrochemical cell further comprises a cell layer. In aspects, the cell layer comprises a liquid-holding cell capable of holding liquid. In aspects, the cell layer comprising the first electrode and the second electrode. In aspects, the liquid-holding cell comprises the first electrode and the second electrode. In aspects, the liquid-holding cell comprises a bottom portion which comprising the first electrode and the second electrode. In aspects, the liquid-holding cell is a flow cell. In aspects, the flow cell comprises an inlet port and an outlet port within the cell layer. In aspects, the cell layer comprises a non-conducting material. In aspects, the cell layer comprises an acrylic polymer and an acrylic copolymer. In aspects, the cell layer comprises an acrylic polymer. In aspects, the cell layer comprises an acrylic copolymer. In aspects, the cell layer comprises poly(methylmethacrylate).

In embodiments, the first electrode and the second electrode are adjacent to a solid support. In aspects, the solid support comprises a non-conducting material. In aspects, the solid support comprises glass.

In embodiments, the biosensor comprises an electrochemical cell as described herein and a sample (e.g. a biological sample). In aspects, the biosensor comprises an electrochemical cell as described herein and a biological sample. In aspects, the biological sample is blood, urine, saliva, lacrimal fluid, nipple aspirate fluid, or cerebrospinal fluid. In aspects, the biological sample is blood. In aspects, the biological sample is urine. In aspects, the biological sample is saliva. In aspects, the biological sample is lacrimal fluid. In aspects, the biological sample is nipple aspirate fluid. In aspects, the biological sample is cerebrospinal fluid.

In embodiments, the disclosure provides methods of detecting a molecule in a sample (e.g. a biological sample) by contacting the electrochemical cell with the sample, thereby detecting the molecule in the sample. In aspects, the methods comprise contacting the electrodes in the electrochemical cell with a sample, and measuring the current of the sample, thereby detecting the molecule in the sample. In aspects, the current of the sample is measured by electrochemical impedance spectroscopy. In aspects, the methods comprise comparing the current measured by electrochemical impedance spectroscopy to a control. Any biomolecules can be detected by the methods described herein, and the skilled artisan can select a ligand appropriate for the biomolecule that is to be detected.

In aspects, the disclosure provides methods of detecting a biomolecule in a liquid sample by contacting the electrochemical cell with the liquid sample, thereby detecting the biomolecule in the liquid sample. In aspects, the methods comprise contacting the electrodes in the electrochemical cell with the liquid sample, and measuring the current of the liquid sample, thereby detecting the biomolecule in the liquid sample. In aspects, the current of the liquid sample is measured by electrochemical impedance spectroscopy. In aspects, the methods comprise comparing the current measured by electrochemical impedance spectroscopy to a control. In aspects, the liquid sample is added to the inlet of the electrochemical cell. In aspects, the liquid sample is a biological sample. In aspects, the biological sample is blood, urine, saliva, lacrimal fluid, nipple aspirate fluid, or cerebrospinal fluid. In aspects, the biological sample is blood. In aspects, the biological sample is urine. In aspects, the biological sample is saliva. In aspects, the biological sample is lacrimal fluid. In aspects, the biological sample is nipple aspirate fluid. In aspects, the biological sample is cerebrospinal fluid. Any biomolecules can be detected by the methods described herein, and the skilled artisan can select a ligand appropriate for the biomolecule that is to be detected. In aspects, the biomolecule is a cancer cell marker. In aspects, the biomolecule is human serum albumin.

In embodiments, the disclosure provides methods of detecting a molecule in a sample by contacting the biosensor with the sample, thereby detecting the molecule in the sample. In aspects, the methods comprise contacting the biosensor with a sample, and measuring the current of the sample, thereby detecting the molecule in the sample. In aspects, the current of the sample is measured by electrochemical impedance spectroscopy. In aspects, the methods comprise comparing the current measured by electrochemical impedance spectroscopy to a control. Any biomolecules can be detected by the methods described herein, and the skilled artisan can select a ligand appropriate for the biomolecule that is to be detected.

In aspects, the disclosure provides methods of detecting a biomolecule in a liquid sample by contacting the biosensor with the liquid sample, thereby detecting the biomolecule in the liquid sample. In aspects, the methods comprise contacting the biosensor with the liquid sample, and measuring the current of the liquid sample, thereby detecting the biomolecule in the liquid sample. In aspects, the current of the liquid sample is measured by electrochemical impedance spectroscopy. In aspects, the methods comprise comparing the current measured by electrochemical impedance spectroscopy to a control. In aspects, the liquid sample is added to the inlet of the biosensor. In aspects, the liquid sample is a biological sample. In aspects, the biological sample is blood, urine, saliva, lacrimal fluid, nipple aspirate fluid, or cerebrospinal fluid. In aspects, the biological sample is blood. In aspects, the biological sample is urine. In aspects, the biological sample is saliva. In aspects, the biological sample is lacrimal fluid. In aspects, the biological sample is nipple aspirate fluid. In aspects, the biological sample is cerebrospinal fluid. Any biomolecules can be detected by the methods described herein, and the skilled artisan can select a ligand appropriate for the biomolecule that is to be detected. In aspects, the biomolecule is a cancer cell marker. In aspects, the biomolecule is human serum albumin.

In embodiments, the disclosure provides methods of forming a modified biosensor with increased sensitivity by modifying a biosensor by: (i) decreasing the thickness of the first electronically conductive polymer, (ii) increasing the recombinant viral surface receptor copy number, or (iii) decreasing the thickness of the first electronically conductive polymer and increasing the recombinant viral surface receptor copy number; thereby forming a modified biosensor with increased sensitivity relative to the unmodified biosensor. The methods further comprise detecting a biomolecule in a biological sample using the modified biosensor. In aspects, the methods comprise forming a modified biosensor with increased sensitivity by (i) detecting a biomolecule in a sample using a biosensor described herein; and (ii) modifying the biosensor by: (a) decreasing the thickness of the first electronically conductive polymer, (b) increasing the recombinant viral surface receptor copy number, or (c) decreasing the thickness of the first electronically conductive polymer, and increasing the recombinant viral surface receptor copy number; thereby forming a modified biosensor with increased sensitivity relative to the unmodified biosensor. In aspects, the recombinant viral surface receptor in the modified biosensor is expressed from a recombinant nucleotide sequence comprising an inducible promoter. In aspects, increasing the recombinant viral surface receptor copy number is accomplished by increasing the amount of inducing agent capable of inducing the inducible promoter relative to the amount of inducing agent used to produce the biosensor.

In embodiments, the disclosure provides methods of forming a modified biosensor with decreased sensitivity by: (i) increasing the thickness of the first electronically conductive polymer, (ii) decreasing the recombinant viral surface receptor copy number, or (iii) increasing the thickness of the first electronically conductive polymer, and decreasing the recombinant viral surface receptor copy number; thereby forming a modified biosensor with decreased sensitivity relative to the unmodified biosensor. The methods further comprise detecting a biomolecule in a biological sample using the modified biosensor. In aspects, the methods comprise forming a modified biosensor with decreased sensitivity by (i) detecting a biomolecule in a sample using a biosensor described herein; and (ii) modifying the biosensor by: (a) increasing the thickness of the first electronically conductive polymer, (b) decreasing the recombinant viral surface receptor copy number, or (c) increasing the thickness of the first electronically conductive polymer and decreasing the recombinant viral surface receptor copy number; thereby forming a modified biosensor with decreased sensitivity relative to the unmodified biosensor. In aspects, the recombinant viral surface receptor in the modified biosensor is expressed from a recombinant nucleotide sequence comprising an inducible promoter. In aspects, decreasing the recombinant viral surface receptor copy number is accomplished by decreasing the amount of inducing agent capable of inducing the inducible promoter relative to the amount of inducing agent used to produce the biosensor.

Embodiments

Embodiment 1. An electrochemical cell comprising: (a) a potentiostat electronically connecting a first electrode and a second electrode; (b) a first electronically conductive polymer between the first electrode and the second electrode; and (c) a viral composition layer above the electronically conductive polymer, the viral composition layer comprising: (i) a whole viral particle comprising a recombinant viral surface receptor; and (ii) a second electronically conductive polymer.

Embodiment 2. The electrochemical cell of Embodiment 1, wherein the first electronically conductive polymer is poly(3,4-ethylenedioxythiophene) polystyrene sulfonate (PEDOT:PSS).

Embodiment 3. The electrochemical cell of Embodiment 1 or 2, wherein the first electronically conductive polymer is a carbon polymer.

Embodiment 4. The electrochemical cell of any one of Embodiments 1 to 3, wherein the first electronically conductive polymer has a resistance from about 0.5 kOhm to about 2.5 kOhm.

Embodiment 5. The electrochemical cell of any one of Embodiments 1 to 4, wherein the first electrode and the second electrode are separated by a space of about 1.5 millimeters.

Embodiment 6. The electrochemical cell of any one of Embodiments 1 to 5, wherein the whole viral particle is embedded within the second electronically conductive polymer.

Embodiment 7. The electrochemical cell of any one of Embodiments 1 to 6, wherein the electrochemical cell comprises a plurality of the whole viral particles within the viral composition layer.

Embodiment 8. The electrochemical cell of any one of Embodiments 1 to 7, wherein the viral composition layer is above the first electrode and the second electrode.

Embodiment 9. The electrochemical cell of any one of Embodiments 1 to 8, wherein the second electronically conductive polymer comprises poly(3,4-ethylenedioxythiophene).

Embodiment 10. The electrochemical cell of any one of Embodiments 1 to 9, wherein the whole virus particle is a M13 filamentous virus particle.

Embodiment 11. The electrochemical cell of any one of Embodiments 1 to 10, wherein the recombinant viral surface receptor is expressed from a recombinant nucleotide sequence comprising an inducible promoter.

Embodiment 12. The electrochemical cell of any one of Embodiments 1 to 11, wherein the recombinant viral surface receptor is capable of binding to a cell surface marker.

Embodiment 13. The electrochemical cell of any one of Embodiments 1 to 12, wherein the recombinant viral surface receptor is capable of binding to a cancer cell surface marker.

Embodiment 14. The electrochemical cell of any one of Embodiments 1 to 13, wherein the recombinant viral surface receptor is capable of binding to a hormone, cytokine, protein, nucleic acid, lipid or carbohydrate.

Embodiment 15. The electrochemical cell of one of Embodiments 1 to 14, further comprising a cell layer forming a liquid-holding cell capable of holding liquid; wherein the liquid-holding cell comprises a bottom portion comprising the first electrode and the second electrode.

Embodiment 16. The electrochemical cell of Embodiment 15, wherein the liquid-holding cell is a flow cell comprising an inlet port and an outlet port within the cell layer.

Embodiment 17. The electrochemical cell of one of Embodiments 1 to 16, wherein the first electrode and the second electrode comprise a metal or carbon.

Embodiment 18. The electrochemical cell of one of Embodiments 1 to 16, wherein the first electrode and the second electrode comprise gold, platinum, silver, palladium, rhodium, lead, copper, or zinc.

Embodiment 19. The electrochemical cell of one of Embodiments 1 to 18, wherein the first electrode and the second electrode are adjacent to a solid support.

Embodiment 20. The electrochemical cell of Embodiment 19, wherein the solid support comprises a non-conducting material.

Embodiment 21. The electrochemical cell of Embodiment 19, wherein the solid support comprises glass.

Embodiment 22. The electrochemical cell of one of Embodiments 15 to 21, wherein the cell layer comprises a non-conducting material.

Embodiment 23. The electrochemical cell of one of Embodiments 15 to 22, wherein the cell layer comprises an acrylic polymer or an acrylic copolymer.

Embodiment 24. The electrochemical cell of one of Embodiments 15 to 23, wherein the cell layer comprises poly(methylmethacrylate).

Embodiment 25. A biosensor comprising the electrochemical cell of any one of Embodiments 1 to 24.

Embodiment 26. The biosensor of Embodiment 25, further comprising a biological sample.

Embodiment 27. The biosensor of Embodiment 26, wherein the biological sample is blood, urine, saliva, lacrimal fluid, nipple aspirate fluid, or cerebrospinal fluid.

Embodiment 28. A method of detecting a biomolecule in a sample, the method comprising: (i) contacting the first electrode and the second electrode of the electrochemical cell of any one of Embodiments 1 to 24 with the sample; (ii) measuring the current of the sample, thereby detecting the biomolecule in the sample.

Embodiment 29. The method of Embodiment 28, wherein the current is measured by electrochemical impedance spectroscopy

Embodiment 30. The method of Embodiment 28 or 29, further comprising comparing the current to a control.

Embodiment 31. The method of any one of Embodiments 28 to 30, wherein the sample is a biological sample.

Embodiment 32. The method of Embodiment 31, wherein the biological sample is blood, urine, saliva, lacrimal fluid, nipple aspirate fluid, or cerebrospinal fluid.

Embodiment 33. The method of Embodiment 31, wherein the biological sample is urine.

Embodiment 34. The method of any one of Embodiments 28 to 33, wherein the biomolecule is a cancer cell marker.

Embodiment 35. The method of any one of Embodiments 28 to 34, wherein the biomolecule is human serum albumin.

Embodiment 36. A diagnostic kit comprising the electrochemical cell of any one of Embodiments 1 to 24 and instructions for use.

Embodiment 37. A method of forming a modified biosensor with increased sensitivity, the method comprising: (i) detecting a biomolecule in a sample using the biosensor of one of Embodiments 25 to 27; and (ii) modifying the biosensor by decreasing the thickness of the first electronically conductive polymer and/or increasing the recombinant viral surface receptor copy number; thereby forming a modified biosensor with increased sensitivity relative to the biosensor.

Embodiment 38. A method of forming a modified biosensor with decreased sensitivity, the method comprising: (i) detecting a biomolecule in a sample using the biosensor of one of Embodiments 25 to 27; and (ii) modifying the biosensor by increasing the thickness of the first electronically conductive polymer and/or decreasing the recombinant viral surface receptor copy number; thereby forming a modified biosensor with decreased sensitivity relative to the biosensor.

Embodiment 39. The method of Embodiment 37 or 38, wherein the recombinant viral surface receptor in the modified biosensor is expressed from a recombinant nucleotide sequence comprising an inducible promoter.

Embodiment 40. The method of Embodiment 39, wherein the increasing the recombinant viral surface receptor copy number is accomplished by increasing the amount of inducing agent capable of inducing the inducible promoter relative to the amount of inducing agent used to produce the biosensor.

Embodiment 41. The method of Embodiment 39, wherein the decreasing the recombinant viral surface receptor copy number is accomplished by decreasing the amount of inducing agent capable of inducing the inducible promoter relative to the amount of inducing agent used to produce the biosensor.

Embodiment 42. A diagnostic kit comprising the biosensor of Embodiment 25.

Embodiment 43. A method of detecting a biomolecule in a sample, the method comprising: (i) contacting the biosensor of Embodiment 25 with the sample; and (ii) measuring the current of the sample, thereby detecting the biomolecule in the sample.

Embodiment 44. The method of Embodiment 43, wherein the current is measured by electrochemical impedance spectroscopy

Embodiment 45. The method of Embodiment 43 or 44, further comprising comparing the current to a control.

Embodiment 46. The method of any one of Embodiments 43 to 45, wherein the sample is a biological sample.

Embodiment 47. The method of Embodiment 46, wherein the biological sample is blood, urine, saliva, lacrimal fluid, nipple aspirate fluid, or cerebrospinal fluid.

Embodiment 48. The method of Embodiment 47, wherein the biological sample is urine.

Embodiment 49. The method of any one of Embodiments 43 to 48, wherein the biomolecule is a cancer cell marker.

Embodiment 50. The method of any one of Embodiments 43 to 49, wherein the biomolecule is human serum albumin.

### EXAMPLES

The examples are for purposes of illustration only and are not intended to limit the scope of the disclosure or claims.

### Example 1: The Virus Bioresistor: Wiring Virus Particles For the Direct, Label-Free Detection of Target Proteins

The virus bioresistor (VBR) is a chemiresistor that directly transfers information from virus particles to an electrical circuit. Specifically, the VBR enables the label-free detection of a target protein that is recognized and bound by filamentous M13 virus particles, each with dimensions of 6 nm (width)×1 μm (length), entrained in an ultra-thin (≈2250 nm) composite virus-polymer resistor. Signal produced by the specific binding of virus to target molecules is monitored using the electrical impedance of the VBR: The VBR presents a complex impedance that is modeled by an equivalent circuit containing just three circuit elements: a solution resistance (Rsoln), a channel resistance (R_{VBR}), and an interfacial capacitance (C_{VBR}). The value of R_{VBR}, measured across five orders of magnitude in frequency, is increased by the specific recognition and binding of a target protein to the virus particles in the resistor, producing a signal ΔR_{VBR}. The VBR concept is demonstrated using a model system in which human serum albumin (HSA, 66 kDa) is detected in a phosphate buffer solution. The VBR cleanly discriminates between a change in the electrical resistance of the buffer, measured by Rₛₒₗₙ, and selective binding of HSA to virus particles, measured by R_{VBR}. The ΔR_{VBR} induced by HSA binding is as high as 200Ω contributing to low sensor-to-sensor coefficients-of-variation (<15%) across the entire calibration curve for HSA from 7.5 nM to 900 nM. The response time for the VBR is 3 to 30 seconds.

Investigating the electrical properties of microscopic biological entities such as organelles, bacteria, eukaryotic cells, and viruses is both interesting from a fundamental science perspective, as well as challenging because they are electrically insulating. How does one “wire” such structures to an external circuit? See Simon et al, Chem. Rev., 116:13009-13041 (2016); Lanzani, Nat. Mat., 13:775-776 (2014); Liao et al, Adv. Mat., 27:7493-7527 (2015). Elegant solutions to this problem have been demonstrated involving interfaces to single cells, bacteria etc. involving single nanostructures or ensembles of nanostructures (nanowires, nanotubes, nanosheets, etc.). For example, electrical signals from single cells have been measured using graphene field-effect transistors, and nanowire-embedded n-p junctions. See Cohen-Karni et al, Nano Lett, 10:1098-1102 (2010); Tzahi Cohen-Karni et al, Nano Lett., 12:2639-2644 (2012). The “wiring” of bacteria to electrode surfaces has been accomplished using outer sphere redox mediators. See Pankratova et al, Electrochem. Commun., 75:56-59 (2017); Yuan et al, Bioelectrochem., 8-12 (2016); Kaneko et al, Bioelectrochem., 114:8-12 (2017).

A new approach, the virus bioresistor provides the means for incorporating virus particles into an electrical circuit (FIG. 1). The VBR has an electronically conductive channel composed of poly(3,4-ethylenedioxythiophene) or PEDOT into which M13 virus particles are embedded (FIG. 1A). Individual M13 virus particles are filamentous with dimensions of 6 nm (width)×1.0 μm (length). The recognition and binding of target molecules to thousands of M13 virus particles embedded in this polymeric channel is signaled by an electrical impedance signature, which can be measured by an external circuit (FIGS. 1B-1C). The impedance response of the VBR is modeled by a simple equivalent circuit containing just three circuit elements: A solution resistance (Rₛₒₗₙ), a channel resistance (R_{VBR}), and an interfacial capacitance (C_{VBR}) (FIG. 17). Information on target binding is contained in the R_{VBR}, which can be measured either at a single frequency or from the best fit of the Nyquist plot across 40 or 50 discrete frequencies using this equivalent circuit.

We demonstrate the VBR concept using a model system in which human serum albumin (HSA, 66 kDa) is detected in a phosphate buffer solution. The VBRs described here have a baseline dc resistance of 200-250Ω which is the same in air or in an aqueous buffer solution, and are capable of producing large signals (ΔR_{VBR}≈250Ω, or ΔR_{VBR}/Rₒ≈100%) for the detection of HSA in phosphate buffer solutions across the entire HSA binding curve ranging from [HSA]=7.5 to 900 nM. In spite of the fact that the electrical signal generated by VBRs derives purely from ensembles of biological entities, extremely high sensor-to-sensor reproducibility of this signal is attainable for the response of VBR biosensors culminating in a coefficient-of-variation of the measured [HSA] for 20 sensors less than 15% across the entire HSA binding curve. The VBR achieves these metrics using a two-terminal, monolithic device architecture that is simple, robust, manufacturable, and inexpensive. No reagents and no sandwich amplification of the impedance signal are required, and no redox species are added to the test solution. Collectively, these data demonstrate that VBR can be used for rapid, inexpensive urine and blood-based assays at the point-of-care.

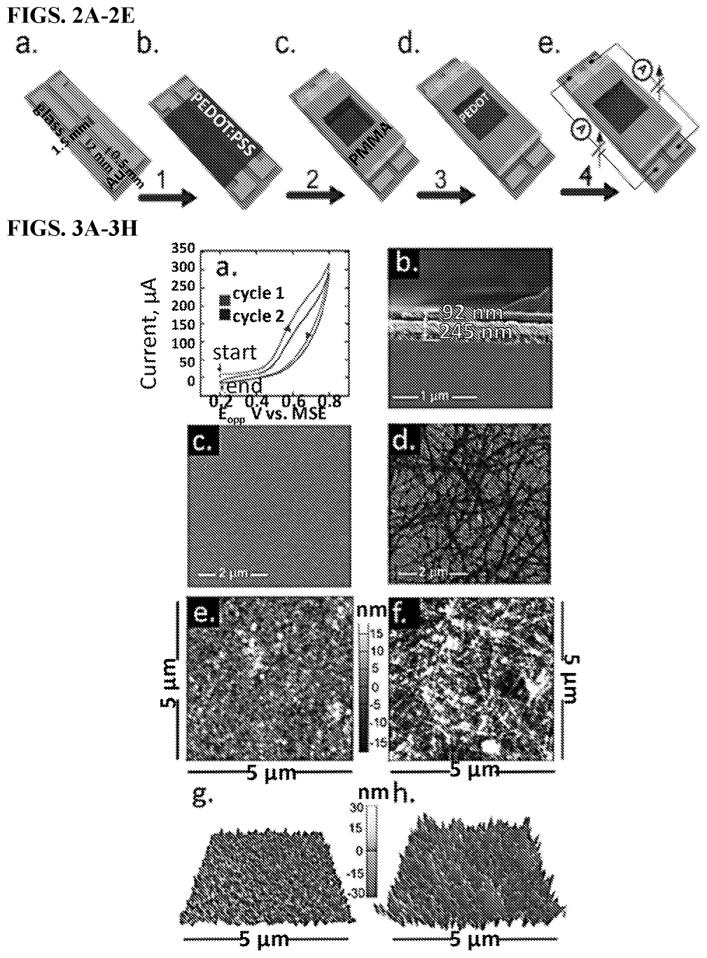

The fabrication of a VBR involves the preparation of two gold electrical contacts on a glass substrate by photolithography (FIG. 2). On top of these contacts, a two-layer VBR channel (15 mm (l)×20 mm (w)) is prepared consisting of a spin-cast PEDOT-PSS semiconductor bottom layer (200-300 nm in thickness) and an electrodeposited virus-PEDOT composite top layer containing thousands of engineered M13 virus particles (90 to 100 nm in thickness). See Donavan et al, Langmuir, 28:12581-12587 (2012); Arter et al, Anal. Chem., 84:2776-2783 (2012); Donavan et al, Anal. Chem., 83:2420-2424 (2011). This virus-PEDOT electrodeposition process involves the application of two oxidizing voltammetric scans to an aqueous solution containing 8 nM M13 virus particles in 12.5 mM LiClO₄, 2.5 mM EDOT (FIG. 3A).

A cross-sectional SEM image of a VBR biosensor film shows a virus-PEDOT top layer with a thickness of about 92 nm on top of about 245 nm PEDOT:PSS bottom layer (FIG. 3B). Plain-view SEMs of pure PEDOT films prepared in an aqueous plating solution of 2.5 mM EDOT and 12.5 mM LiClO₄ show a smooth, homogenous surface (FIG. 3C). Virus-PEDOT films prepared from the same plating solution with the addition of 8 nM virus show dark, filamentous structures within the virus-PEDOT top layer (FIG. 3D). These filaments are M13 bacteriophage, which have typical dimensions of 6 nm (diameter)×1.0 μm (length). Atomic force microscopy (AFM) images show that in the absence of virus particles, the virus-PEDOT top layer is smooth with an RMS surface roughness of 5 nm (FIGS. 3E, 3G). If this layer is produced to contain virus particles, a slightly rougher surface is seen with an RMS roughness of 10 nm; however, a distinct topography reveals the presence of fiber like structures that can be attributed to PEDOT-covered virus strands protruding from the PEDOT surface (FIGS. 3F, 3H). After the virus-PEDOT top layer is electrodeposited, the bioaffinity layer is complete, and the VBR is ready to use.

Analytical equations for the real and imaginary components of the complex impedance, Zᵣₑ and Zᵢₘ (FIG. 17), are used to fit experimental impedance data to extract the values of the three circuit elements: Rₛₒₗₙ, R_{VBR}, and C_{VBR}. A version of the equivalent circuit in which a constant phase element (CPE) is substituted for each capacitor is used for this purpose. This elaboration provides better agreement between the calculated and the experimental impedance data, resulting in improved precision for the measurement of R_{VBR} (FIG. 17). The impedance of a CPE, Z_{CPE}, and the capacitive impedance, Z_{C}, are defined by these equations:

$${Z}_{C} = \frac{1}{i ω C} {Z}_{C P E} = \frac{1}{i ω {Q}^{n}}$$

where ω is the angular frequency (s⁻¹), i=√(−1). Qⁿ is the CPE capacitance (F) where n has a value of 1.0 if the CPE is purely capacitive. n is used as a fitting parameter in this study and has a value of 1.0<n<1.2.

The VBR produces a distinctive impedance response consisting of a semicircular Nyquist plot (Zᵢₘ versus Zᵣₑ as a function of frequency) (FIGS. 4A-4C). This response resembles the Randles equivalent circuit that is commonly seen for electrochemical biosensors operating in the presence of an added redox species, such as Fe(CN)₆^{3-/4-}. See Yu et al, Food Chem., 176:22-26 (2015); Eissa et al, Biosen. Bioelectron., 69:148-154 (2015). The semicircular Nyquist plot for electrochemical biosensors derives from electron transfer to and from the redox species present in the solution. When a redox species is not added, no semicircle is observed. The VBR produces a semicircular Nyquist plot without added redox species. Instead, the VBR channel presents a parallel resistance (dominated by electron conduction through the polymer composite VBR) and capacitance (produced by the non-Faradaic charging and discharging of the electrical double layer at the surface of the VBR). The semicircular Nyquist plots aids in the precision with which R_{VBR} can be measured—just as it does in electrochemical biosensors that use the diameter of this semicircle—the so-called charge transfer resistance—to transduce target binding. See Zhang et al, Biosens. Bioelectron., 75:452-457 (2016); Li et al, Anal. Chem., 84(8):3485-3488 (2012); Gao et al, Anal. Chem., 85(3):1624-1630 (2013).

VBR biosensors are able to distinguish between changes in the electrical resistance of the test solution, caused by variations in the salt concentration for example, and the concentration of target molecules present in this solution. Information on the electrical conductivity of the solution is contained in Rₛₒₗₙ whereas the concentration of target protein is encoded by R_{VBR}. Virtually no cross-talk occurs between these two circuit elements. For example, Nyquist plots (Zᵢₘ versus Zᵣₑ as a function of frequency) for a VBR in three concentrations of PBS buffer (1×PBS, 2.5×PBS and 5×PBS) show the same ΔR_{VBR}=R_{VBR,HSA}−R_{VBR,buffer} signal for 75 nM HSA (FIG. 4E) independent of the salt concentration ([NaCl]) over the range of 134 to 670 mM. Notably, Rₛₒₗₙ decreases dramatically with increasing salt concentration (FIG. 4D).

The complementary experiment is to vary [HSA] in a 1×PBS buffer solution (FIG. 4F). Here, Nyquist plots are shown for five buffer solutions containing [HSA]=0 nM, 70 nM, 220 nM, 370 nM, and 750 nM. In this case, a quasi-linear increase in ΔR_{VBR} with [HSA] is measured (FIG. 4H), and Rₛₒₗₙ remains constant (FIG. 4G). This property of VBRs—the ability to parse changes in impedance due to the solution resistance and target binding—provides an enormous advantage in terms of the application of this biosensor technology to body fluids where salt concentrations are unknown and uncontrolled.

VBR performance was evaluated for the detection of HSA using 20 VBRs in order to assess sensor-to-sensor reproducibility and coefficient-of-variance (CoV) to determine their practicality for single use biosensors. Two methods for analyzing VBR impedance data are also assessed here. The first method was previously used for non-faradaic impedance biosensors where the signal-to-noise guided the selection of a single frequency at which either ΔZᵢₘ or ΔZᵣₑ was calculated by, for example, Z_{re,HSA-Z}^{o}ᵣₑ. See Ogata et al, Anal. Chem., 89:1373-1381 (2017). Using this approach, the sensing signal at 5 Hz was selected. The second method exploits a range of impedance data across 40-50 discrete frequencies and employs a fit to the equations of FIG. 17 to determine ΔR_{VBR}. Method 1 will afford more rapid analysis because impedance data at a single frequency is required. Method 2 requires longer analysis times; however, the approach has the potential to provide for higher precision and reduced noise for an assay. The two methods were compared for three independent VBR biosensors (N=3) at each HSA concentration from 7.5 nM to 750 nM to evaluate sensor-to-sensor reproducibility. In addition, two sensors (N=2) were tested at 900 nM [HSA].

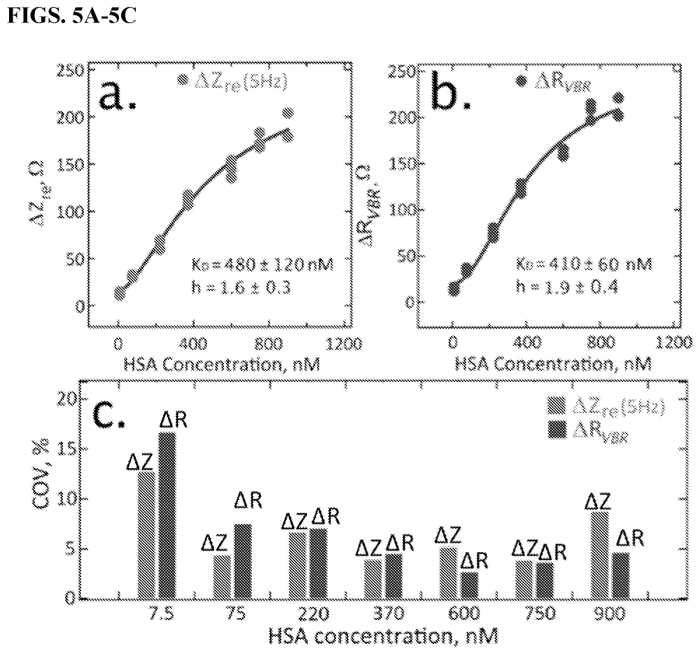

The performance of Methods 1 and 2 are summarized in the plots of FIG. 5A and FIG. 5B, respectively. There is little difference in the performance of these two methods in terms of sensitivity, precision, and noise. Both ΔZ_{re, 5 Hz} (Method 1) and ΔR_{VBR} (Method 2) track increases in the HSA concentration from 7.5 nM to 900 nM HSA, saturating at close to 900 nM. These two calibration plots are both fitted with the Hill equation, which is frequently used to model biosensor response (Xia et al, ACS Synth. Biol., 6(10):1807-1815 (2017)):

$$Δ {Z}_{re} = Δ {Z}_{re , lim} + \frac{Δ {Z}_{re , 0} - {ΔZ}_{re , lim}}{1 + {( \frac{{C}_{HSA}}{{K}_{D}} )}^{h}}$$

The best fit to the Hill equation for the ΔZᵣₑ calibration plot results in ΔZ_{./,1&2}=250±40Ω, ΔZ_{./,4}=16±5Ω, K=480±120 nM, h=1.6±0.3, and R²=0.97. Fit to the Hill equation for the ΔR_{channel} calibration plot results in ΔR_{ABC,1&2}=250±30Ω, ΔR^{o}_{VBR}=20±5Ω, K=410±60 nM, h=1.9±0.3, and R²=0.98. These data provide no justification for the use of multiple analysis frequencies (Method 2) as compared with a single, S/N-selected, analysis frequency (Method 1). Apparent K_{D} values are identical within experimental error. Values of h, which indexes the degree of cooperativity in target binding to virus particles, are also identical and equal to 1.6, which indicates significant cooperativity for phage binding to HSA in this system.

The origin of the VBR impedance signal is of interest, and remains the subject of investigation. Either of two signal transduction mechanisms could reasonably account for our observations: First, the PEDOT-PSS can function as a p-type organic semiconductor field effect transistor (FET). See Gao et al, Anal. Chem., 85(3):1624-1630 (2013); Chu et al, Sci. Rep., 7(1) (2017). In this case, an increase in ΔR_{VBR} with [HSA] is accounted for by the binding of a positively charged target molecule to the VBR, leading to depletion of majority carriers and an increase in impedance. But HSA has an isoelectric point, pI=5.3 (Dockal, M.; Carter, D. C.; Ru, F. October 1999, 274 (41), 29303-29310), and our PBS buffer has pH=8.0. So, the analyte in these experiments is expected to have an overall negative charge, not a positive charge, at this pH. The binding of HSA to the PEDOT VBR should therefore cause the accumulation of majority carriers, reducing its electrical impedance, which is contrary to our experimental observations. As shown in FIG. 4E, the signal amplitude observed for HSA is unaffected by increases in the salt concentration of the test solution from 1×PBS to 5×PBS. This indicates that an electric field effect is not involved in the signal transduction process, since the Debye length in these buffer solutions is both very small (2-8 Å) and variable.

A second, previously observed mechanism involves the disruption of long range ordering in the PEDOT-PSS polymer chains. For example, bulky intercalators such as tosylate anions can cause an increase in electrical resistance (Meier et al, J. Phys. Chem. C, 120:21114-21122 (2016)), or secondary dopants (e.g., diethylene glycol, polyethylene glycol, dimethyl sulfoxide, and the like) that lubricate the motion of polymer chains thereby promoting a higher degree of long range ordering and a lower electrical resistance. HSA is readily classified as falling into the first category of bulky, structure disrupter. This description qualitatively explains the increases in resistance seen for VBRs upon exposure to HSA reported here. Furthermore, this model is consistent with the observed impedance signal for HSA measured at VBRs remaining unrelated to the salt concentration of the test solution.

In addition to sensitivity and reproducibility, selectivity and speed are the two other attributes important for biosensors. The selectivity of VBR biosensors was examined with two control conditions: (1) a VBR virus-PEDOT film containing HSA-binding virus measured for binding to 750 nM BSA protein, which is closely matched to HSA in terms of both size (both 66.5 kDa) and amino acid sequence (76% homologous) (Majorek et al, Mol Immunol., 52:174-182 (2012)), and (2) a VBR virus-PEDOT film containing the negative control STOP4 virus, which has no displayed peptide ligands, in the presence of 750 nM HSA protein. The sensing signal is described as ΔR_{VBR}=R_{VBR,HSA}−R_{VBR,PBS}, determined by fitting the impedance data with the equivalent circuit of FIG. 17. Both control VBR biosensors show less than ˜1Ω in of change (in either ΔR_{VBR} or ΔZᵣₑ) in comparison to ˜200Ω resistance increase for HSA-virus-PEDOT films against 750 nM HSA. The impedance response for VBRs gives excellent binding signal specific to HSA at 200× over background (FIG. 6A). Real-time VBR measurements (FIG. 6B) allow the response time of these devices to be directly measured. We observe a rapid (3-30 s) step-wise increase in ΔZᵣₑ followed by near instantaneous settling of Z_{Re} at the concentration-appropriate value (FIG. 6B). This constitutes a near ideal response function for a biosensor and demonstrates the potential utility of VBRs for point-of-care applications.

The VBR simplifies the problem of electrically communicating with virus particles, and importantly, extracting valuable information in this process. Communication takes the form of an increase in the electrical impedance of the virus-PEDOT VBR in the presence of a target protein disease marker, relative to the impedance measured in a pure buffer solution. This impedance increase of up to 200Ω signals the degree to which virus-displayed peptides have recognized and bound a particular target protein, leading to precise and highly reproducible measurement of the concentration of this target molecule. The VBR is able to by-pass a ubiquitous noise source in electrical or electrochemical biosensing: the variable electrical impedance of the solution itself

### Example 2: Detection of DJ-1 Bladder Cancer Biomarker With the VBR

The VBR successfully detected a wide range of concentration for HSA (human serum albumin) protein with 8 nM L3 phage loaded into the PEDOT film of the sensor. To test the diverse applicability in terms of protein detection, DL-1 phage was incorporated into the sensor for the detection of DJ-1 bladder cancer biomarker. DJ-1 is a ˜20 kDa protein as compared to HSA, a 66 kDa protein detected in Example 1.

VBRs were fabricated with some parameters imposed on each step of fabrication (FIG. 8). The VBRs for DJ-1 were subjected to sensing experiments with the baseline reading in synthetic urine (step 5 of FIG. 8). In an effort to generate a higher signal and achieve lower detection limits, changes were introduced in step 2; wherein the base layer of baked PEDOT:PSS was spin-coated to yield a range of DC resistances across the electrodes. Sensor fabrication remained the same for all steps that followed (See, Bhasin et al.). The DL-1 phage was incorporated into the PEDOT layer by electrochemical entrapment.

To study the effect of increasing DC resistance of PEDOT:PSS base layer on the overall signal generated by VBR, many sensors were fabricated with a base layer DC resistance 74-360Ω and were exposed to 100 nM DJ1 protein. All sensors were loaded with 8 nM DL1 phage. It was hypothesized that as the base layer resistance is increased, more current is forced through the PEDOT:phage layer thereby generating higher signal. It was concluded that increasing the base layer resistance increases the signal by orders of magnitude, and that the highest signal is generated in the 240-360Ω DC resistance range.

To expose VBR to different concentrations of DJ1 protein, many sensors were fabricated with a base layer 240-360Ω DC resistance and were exposed to different concentrations of DJ1 protein. All sensors were loaded with 8 nM DL1 phage. It was concluded that the VBR can distinguish between different concentrations of DJ1 protein; that the strategy to increase the base layer resistance yielded limit of detection is in pico-molar range, 10 pM as compared to 7 nM detected in VBR for HSA protein; and that the coefficient of variation for the sensors exposed to same concentration is below 14%.

To test the specificity of the VBR, control experiments were conducted with sensors loaded with no phage and exposed to 1000 nM protein (no phage control). Stop4 control phage was loaded into the sensor and tested against 1000 nM protein (Stop4 control). The results were compared with DL1 loaded sensor exposed to 500 nM DJ1 protein. It was concluded that the two control experiments successfully demonstrated the specificity of the sensor. It was noted that increasing the base layer resistance resulted in developing 10 pM sensitivity in the VBR for DJ1 protein as against 7 nM sensitivity displayed by the VBR for HSA protein. It was also noted that a new signal amplification strategy is introduced, wherein DJ1 protein forms a sandwich with two phages, where each phage displays a different specific binder.

To improve the selectivity and sensitivity of the VBR, electrochemical impedance measurements were conducted at different steps of the bioresistor fabrication and detection of DJ1. It was concluded that a significant increase in the impedance after incorporation of phage on top of carbon nanopowder film. It was also concluded that the impedance increases further after incubation with 10 nM of DJ-1 and the second phage DL2.

### Example 3: Propagation of M13 Phage-Displayed Ligands From Phagemids

This example defines the processes for the preparation of phage-displayed polypeptide ligands. FIG. 17 shows the operational flowchart, as described in detail herein.

Equipment and supplies: Disposable baffled flasks with vented closure, 125-250 mL; Thompson Ultra Yield™ Flasks, 500 mL-2.5 L; AirOtop™ Enhanced Seals; Polypropylene centrifuge bottles, 250-500 mL; Quartz Cuvette, 50 μL; Disposable cuvettes; Ice bucket; Polypropylene beaker, sterile, 100-250 mL; 1.5-5 mL polypropylene microcentrifuge tubes; 500 mL polypropylene graduated cylinder; Manual Micropipettes, 0.5 μL-5000 μL; Eppendorf Repeater® M4; Pipette controller; Aerosol barrier, low retention pipette tips, 10 μL-1250 μL, sterile; 1000-5000 μL Macro disposable sterile pipet tips; Eppendorf Combitips advanced®, 25-50 mL; Disposable serological pipets, 5-50 mL; Beckman Avanti J-25 centrifuge; Beckman JA-14 or JA-10 fixed-angle rotor; Cary 60 UV-Visible spectrophotometer; HERMLE Z216MK refrigerated microcentrifuge; HERMLE rotor 220.88/221.35; Eppendorf I26R incubated shaker; 125 mL-2.5 L shake flask clamp; Chlorine-based bleach; Glycerol inoculum stock (E. coli F′⁺ containing phagemid-ligand fusion); M13KO7 Helper phage; 2×YT media, sterile filtered; 50 mg/mL Carbenicillin disodium salt; 5 mg/mL Tetracycline hydrochloride; 40 mg/mL Kanamycin sulfate; 1.0 M Isopropyl-β-D-1-thiogalactopyranoside (IPTG); Milli-Q™ ultrapure water; 20% (w/v) PEG-8000/2.5 M NaCl; Resuspension Buffer (RB): 1×PBS (pH 7.4-8.0), 0.05% (v/v) Tween® 20 (polysorbate 20), 10% (v/v) glycerol; 1×PBS (pH 7.4-8.0).

Propagation of M13 phage-displayed ligands: Pre-warmed an LB-carbenicillin plate at 37±2° C. until any condensation was fully evaporated. Placed cell stock containing F′ strain E. coli carrying the phagemid from the −80° C. freezer into a −20° C. cooling block. Used a sterile pipette tip to jab the cell stock several times. Used the tip to streak the pre-warmed LB-carbenicillin plate. Returned the cell stock to the −80° C. freezer. Incubated the LB-carbenicillin plate overnight at 37±2° C.≥12 hours. Near a flame, prepared primary culture by adding 15 mL 2×YT per 300 mL expression culture to a sterile disposable baffled flask with vented closure. If preparing several expression cultures, added 15 mL+(A additional expression cultures×10 mL)=B mL 2×YT. Added carbenicillin (50 mg/mL) to a final concentration of 50 μg/mL directly to flask. Added tetracycline (5 mg/mL) to a final concentration of 2.5 μg/mL directly to flask. Used a sterile pipette tip to obtain a single colony of F′ strain E. coli carrying the phagemid from the streaked LB-carbenicillin plate and gently swirled the tip in the prepared media. Transferred the culture to an incubated shaker to incubate at 37° C. with shaking at 225 rpm until an OD₆₀₀ of 0.5-0.7 was achieved. Measured and recorded final volume of culture using a sterile serological pipette. Added IPTG to culture to a final concentration of 30 μM. Added M13KO7 helper phage to achieve 99.9% infectivity (MOI=4.6).

$$( \frac{Virion}{Cell} ) ( \frac{1 {0}^{9} cells}{mL } ) ( \frac{V culture mL}{6.022 \times 1 {0}^{23} virions} ) ( \frac{1}{CM 13 K 07 nM} ) ( \frac{1 {0}^{9} nmol}{mol} ) ( \frac{1 {0}^{6} µL}{L} ) = \_VM 13 K 07 ( µL )$$

Returned the culture to the incubated shaker for 45 minutes at 37° C. at 225 rpm. Near a flame, prepared each expression culture by adding desired volume of 2×YT to an Ultra Yield Flask. If the volume of the expression culture was <400 mL, used a 1 L Ultra Yield Flask. For expression culture volumes ≥400 mL, used a 2.5 L Ultra Yield Flask. To each flask, added carbenicillin (50 mg/mL) to a final concentration of 50 μg/mL. To each flask, added kanamycin (40 mg/mL) to a final concentration of 20 μg/mL. To each flask, added IPTG to a final concentration of 30 μM. Transferred 8 mL of primary culture to each expression culture. Covered each flask with AirOtop Enhanced Seal. Transferred each expression culture to an incubated floor-model shaker to incubate for ≥18 hours overnight at 30° C. with shaking at 225 rpm.

Harvesting the M13 phage-displayed peptide ligands from culture: After ≥18 hours of incubation, transferred each culture to two 250 mL autoclaved centrifuge bottles or a single 500 mL centrifuge bottle. Centrifuged the cultures at 15,300×g for ≥10 minutes at 4° C. For each culture, transferred 30 mL of 20% (w/v) PEG-8000/2.5 M NaCl solution to two 250 mL centrifuge bottles or 60 mL to a single 500 mL centrifuge bottle. Incubated centrifuge bottles containing PEG/NaCl on ice. After centrifugation, transferred each supernatant to the centrifuge bottles containing PEG/NaCl. Mixed thoroughly by gently inverting each bottle ten or more times; incubated on ice for ≥30 minutes. Centrifuged at 15,300×g for ≥15 minutes at 4° C. Without disturbing the pellets, decanted and disposed of the supernatant, diluting with bleach to a final concentration of ≥10% (v/v). Returned the bottles to the centrifuge with the pellets facing away from the central axis of the rotor. Then, centrifuged at 2,500×g for ≥4 minutes at 4° C. Removed the bottles from the rotor with the pellets face-up. Carefully transferred the bottles to ice, ensuring the pellets continued to sit face-up. Using a serological pipette, carefully removed the residual supernatant, diluting with bleach to a final concentration of ≥10% (v/v). Added 50 mL resuspension buffer (RB) to one centrifuge bottle, then resuspended the phage pellet with a serological pipette. Transferred the solution to the other centrifuge bottle (if applicable) and resuspended the phage pellet with a serological pipette. Centrifuged at 22,100×g for ≥4 minutes at 4° C. to sediment insoluble debris. Transferred each supernatant to a separate sterile container. Using a repeater pipette, divided each resuspension into 4 mL and 1.5 mL volumes, using 5 mL and 2 mL microcentrifuge tubes, respectively. Labeled microcentrifuge tubes with assigned lot number of phage. Snap-freezed in liquid nitrogen, then stored at −80° C. in box labeled with lot number, analyst's initials and date.

PEG Precipitation of phage-displayed peptide ligands: For every aliquot of phage to undergo precipitation, thawed on ice. Added 20% volume of 20% PEG-8000/2.5 M NaCl solution. X mL phage×0.2=Y mL PEG/NaCl. Mixed thoroughly by inverting each tube ten times; incubate on ice for 30 minutes. Centrifuged at 13,520×g for ≥20 minutes at 4° C. Without disturbing the pellets, decanted and disposed of the supernatant, diluting with bleach to a final concentration of ≥10% (v/v). Returned the tubes to the centrifuge with the pellets facing away from the rotor's central axis, then centrifuged at 1,500×g for ≥4 minutes at 4° C. Disposed of the residual supernatant using a pipette fitted with a filter tip. Re-suspended each precipitate with 1×PBS pH 7.4-8.0, using 25% the original volume of buffer: X mL original phage×0.25=Y mL PBS pH 7.4-8.0. Combined any replicates. Centrifuged the suspension at 13,520×g for ≥4 minutes at 4° C. Transferred the supernatant to a sterile 1.5 or 2.0 mL microcentrifuge tube; labeled the tube with the analyst's initials, date and strain of phage. Created a 10-fold dilution of phage to a final volume of 60 μL. Used ≥50 ≥L of this sample to perform a spectroscopic analysis, measuring the absorbance spectrum from 240-340 nm in a quartz cuvette. With this data, calculated the concentration of phage using the following formula:

C(nM)=Abs₂₆₈×Dilution Factor(10)×8.31 nM

The target absorbance was between 0.1-3.5 at 268 nm. If the absorbance exceeded 3.5, diluted the sample. If the absorbance was below 0.1, used a more concentrated sample. Stored the phage at 4° C.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. An electrochemical cell comprising:
(a) a potentiostat electronically connecting a first electrode and a second electrode;
(b) a first electronically conductive polymer between said first electrode and said second electrode; and
(c) a viral composition layer above said electronically conductive polymer, the viral composition layer comprising:
(i) a whole viral particle comprising a recombinant viral surface receptor; and
(ii) a second electronically conductive polymer.

2. The electrochemical cell of claim 1, wherein said first electronically conductive polymer is poly(3,4-ethylenedioxythiophene) polystyrene sulfonate.

3. The electrochemical cell of claim 1, wherein said first electronically conductive polymer is a carbon polymer.

4. The electrochemical cell of claim 1, wherein the first electronically conductive polymer has a resistance from about 0.5 kOhm to about 2.5 kOhm.

5. The electrochemical cell of claim 1, wherein the first electrode and the second electrode are separated by a space of about 1.5 millimeters.

6. The electrochemical cell of claim 1, wherein said whole viral particle is embedded within said second electronically conductive polymer.

7. The electrochemical cell of claim 1, wherein said electrochemical cell comprises a plurality of said whole viral particles within said viral composition layer.

8. The electrochemical cell of claim 1, wherein said viral composition layer is above said first electrode and said second electrode.

9. The electrochemical cell of claim 1, wherein said second electronically conductive polymer comprises poly(3,4-ethylenedioxythiophene).

10. The electrochemical cell of claim 1, wherein the whole virus particle is a M13 filamentous virus particle.

11. The electrochemical cell of claim 1, wherein the recombinant viral surface receptor is expressed from a recombinant nucleotide sequence comprising an inducible promoter

12. The electrochemical cell of claim 1, wherein the recombinant viral surface receptor is capable of binding to a cell surface marker.

13. The electrochemical cell of claim 1, wherein the recombinant viral surface receptor is capable of binding to a cancer cell surface marker.

14. The electrochemical cell of claim 1, wherein the recombinant viral surface receptor is capable of binding to a hormone, cytokine, protein, nucleic acid, lipid or carbohydrate.

15. The electrochemical cell of claim 1, further comprising a cell layer forming a liquid-holding cell capable of holding liquid; wherein the liquid-holding cell comprises a bottom portion comprising the first electrode and the second electrode.

16. The electrochemical cell of claim 15, wherein the liquid-holding cell is a flow cell comprising an inlet port and an outlet port within the cell layer.

17. The electrochemical cell of claim 1, wherein the first electrode and the second electrode comprise a metal or carbon.

18. The electrochemical cell of claim 1, wherein the first electrode and the second electrode comprise gold, platinum, silver, palladium, rhodium, lead, copper, or zinc.

19. The electrochemical cell of claim 1, wherein the first electrode and the second electrode are adjacent to a solid support.

20. The electrochemical cell of claim 19, wherein the solid support comprises a non-conducting material.

21. The electrochemical cell of claim 19, wherein the solid support comprises glass.

22. The electrochemical cell of claim 15, wherein the cell layer comprises a non-conducting material.

23. The electrochemical cell of claim 15, wherein the cell layer comprises an acrylic polymer or an acrylic copolymer.

24. The electrochemical cell of claim 15, wherein the cell layer comprises poly(methylmethacrylate).

25. A biosensor comprising the electrochemical cell of claim 1.

26. The biosensor of claim 25, further comprising a biological sample.

27. The biosensor of claim 26, wherein the biological sample is blood, urine, saliva, lacrimal fluid, nipple aspirate fluid, or cerebrospinal fluid.

28. A method of detecting a biomolecule in a sample, the method comprising:
(i) contacting the first electrode and the second electrode of the electrochemical cell of claim 1 with the sample; and
(ii) measuring the current of the sample, thereby detecting the biomolecule in the sample.

29. The method of claim 28, wherein the current is measured by electrochemical impedance spectroscopy.

30. The method of claim 28, further comprising comparing the current to a control.

31. The method of claim 28, wherein the sample is a biological sample.

32. The method of claim 31, wherein the biological sample is blood, urine, saliva, lacrimal fluid, nipple aspirate fluid, or cerebrospinal fluid.

33. The method of claim 31, wherein the biological sample is urine.

34. The method of claim 28, wherein the biomolecule is a cancer cell marker.

35. The method of claim 28, wherein the biomolecule is human serum albumin.

36. A diagnostic kit comprising the electrochemical cell of claim 1 and instructions for use.

37. A method of forming a modified biosensor with increased sensitivity, the method comprising:
(i) detecting a biomolecule in a sample using the biosensor of claim 25; and
(ii) modifying said biosensor by decreasing the thickness of said first electronically conductive polymer and/or increasing the recombinant viral surface receptor copy number thereby forming a modified biosensor with increased sensitivity relative to said biosensor.

38. A method of forming a modified biosensor with decreased sensitivity, the method comprising:
(i) detecting a biomolecule in a sample using the biosensor of claim 25; and
(ii) modifying said biosensor by increasing the thickness of said first electronically conductive polymer and/or decreasing the recombinant viral surface receptor copy number thereby forming a modified biosensor with decreased sensitivity relative to said biosensor.

39. The method of claim 37, wherein the recombinant viral surface receptor in said modified biosensor is expressed from a recombinant nucleotide sequence comprising an inducible promoter.

40. The method of claim 37, wherein said increasing the recombinant viral surface receptor copy number is accomplished by increasing the amount of inducing agent capable of inducing said inducible promoter relative to the amount of inducing agent used to produce said biosensor.

41. The method of claim 38, wherein the recombinant viral surface receptor in said modified biosensor is expressed from a recombinant nucleotide sequence comprising an inducible promoter.

42. The method of claim 38, wherein said decreasing the recombinant viral surface receptor copy number is accomplished by decreasing the amount of inducing agent capable of inducing said inducible promoter relative to the amount of inducing agent used to produce said biosensor.

43. A diagnostic kit comprising the biosensor of claim 25.
